(19) United States
(12) Patent Application Publication
(10) Pub. No.: US 2026/0246636 A1
(43) Pub. Date: Aug. 20, 2026

(54) AI AGENT RISK MITIGATION BASED ON DETERMINISTIC FINGERPRINTING

(71) Applicant: RPM-One, Inc. dba Drive Health, Gilbert, AZ (US)

(72) Inventors: Alexander SICULAR, Dallas, TX (US); Kevin LONGORIA, Gilbert, AZ (US); Rajkumar KHANDELWAL, Chandler, AZ (US)

(73) Assignee: RPM-One, Inc. dba Drive Health, Gilbert, AZ (US)

(21) Appl. No.: 19/541,694

(22) Filed: Feb. 17, 2026

Related U.S. Application Data
(60) Provisional application No. 63/760,607, filed on Feb. 19, 2025.

(51) Int. Cl. H04L 9/32; H04L 9/08

(52) U.S. Cl. CPC H04L 9/321; H04L 9/0819

(57) ABSTRACT

Systems and methods for credentialing artificial intelligence (AI) agents are disclosed. A system can receive, by an exchange from an entity providing an AI agent, a request for the AI agent to obtain a digitally verifiable policy credential for coverage of the AI agent. The request can include a deployed agent fingerprint for the AI agent, at least one of a digitally signed verifiable credential or a digitally signed verifiable license of the AI agent, and one or more parameters for the coverage. The system can cryptographically validate the credential or license, determine that a hash of the deployed agent fingerprint matches a hash of the credential or license, and provide an agent data package to a plurality of systems. The system can generate and transmit a digitally verifiable policy credential including the deployed agent fingerprint and a policy corresponding to the coverage parameters.

### CROSS REFERENCE TO RELATED APPLICATIONS

The present application claims the benefit of and priority to U.S. Provisional Application No. 63/760607, filed Feb. 19, 2025, the disclosure of which is incorporated herein by reference in its entirety.

### BACKGROUND

Artificial intelligence (AI) systems, including agentic AI systems, can include numerous interdependent software and data components that interact to perform computational tasks. Ensuring that these components remain authentic, verifiable, and properly bound to the specific deployed instance of the agent can be challenging, particularly when agents are updated, reconfigured, or distributed across heterogeneous systems. For example, entities interacting with an AI agent may depend on policies that assess the probability of operational incidents and implement coverage terms designed to mitigate potential losses associated with performance deviations. The policies may be generated based on artifacts such as verifiable licenses or credentials. However, verifying authenticity and correspondence of the artifacts to a given AI agent may be difficult due to the dynamic and distributed nature of AI deployments.

### SUMMARY

AI agents executing tasks in regulated operational environments can be approved for deployment to execute tasks based on active policies that implement coverage terms designed to mitigate potential losses associated with performance deviations. The policies may be generated based on verifiable credentials and licenses that certify performance of the AI agents. Conventional architectures use static data registries that can result in latency, can limit scalability, and/or can cause data inconsistency when credentials and/or licenses are exchanged between separate systems. For example, static data registries may not verify the credentials and/or the licenses, which may not allow for detection of invalid (e.g., forged, outdated, and/or the like) credentials. As another example, there can be mismatches between the a version of deployed AI agent (e.g., a model version) and the credentials or licenses presented, leading to inaccurate or stale authorizations. Conventional architectures may therefore suffer from insufficient data fidelity when generating policies.

The techniques described herein can provide a unified digital credentialing and policy issuance framework for AI agents using cryptographically verifiable identifiers and structured metadata exchanges. In some implementations, the techniques can employ agent fingerprints that are deterministically derived from component manifests and deployment configurations of a given AI agent. Based on the agent fingerprints, the techniques can verify that a given credential and/or license correspond to the version of the given AI agent. In some implementations, the techniques can use credential and license verification through public key infrastructures combined with automated policy generation workflows. Based on verification through the public key infrastructures, the techniques can verify authenticity of the credential and/or license submitted for the given AI agent. The described architecture can coordinate between credential granting organizations, license granting organizations, and insurance entities using authenticated application programming interfaces to transmit digitally signed credentials, licenses, and policy information. By aligning all exchanged data (e.g., credentials, licenses, and/or policy information) to cryptographically verifiable identifiers, the techniques described herein can maintain a continuous chain of authenticity between the creation of an AI agent and generation of a policy for the AI agent. The techniques described herein can therefore be employed to generate a policy with coverage terms designed to mitigate potential losses arising from performance deviations, utilizing data that is both authentic and accurately linked to the specific version of the AI agent. The AI agent can then be authorized for deployment to execute a task based on the policy.

At least one aspect relates to a system. The system can receive, through an exchange from an entity providing an artificial intelligence (AI) agent, a request for the AI agent to obtain a digitally verifiable policy credential for coverage of the AI agent. The request can include a deployed agent fingerprint for the AI agent, at least one of a digitally signed verifiable credential of the AI agent or a digitally signed verifiable license of the AI agent, and one or more parameters for the coverage. The system can cryptographically validate the at least one of the digitally signed verifiable credential using a public key of a credentialing entity or the digitally signed verifiable license using a public key of a licensing entity. The system can determine that a hash of the deployed agent fingerprint matches a hash of at least one of the digitally signed verifiable credential or the digitally signed verifiable license. The system can provide, to a plurality of systems corresponding to policy credentialing entities, an agent data package that includes the deployed agent fingerprint, the at least one of the digitally signed verifiable credential or the digitally signed verifiable license, and the one or more parameters for the coverage. The system can receive, from the plurality of systems, a plurality of requests to provide the digitally verifiable policy credential. The system can receive a selection of one of the plurality of requests. The system can generate the digitally verifiable policy credential according to the selected request, the digitally verifiable policy credential including the deployed agent fingerprint, a policy corresponding to the one or more parameters, and the digitally verifiable policy credential digitally signed based on a key of the exchange. The system can transmit the digitally verifiable policy credential to the entity.

In some implementations, the system can provide instructions to the entity providing the AI agent to store at least part of the agent data package in a compliance registry. In some implementations, the system can provide instructions for at least one of the plurality of systems to store the agent data package in a database. In some implementations, the system can assign the AI agent to a class of a plurality of classes, the plurality of classes corresponding to levels of incident probability, based on the at least one of the digitally signed verifiable credential of the AI agent or the digitally signed verifiable license of the AI agent received from the entity. In some implementations, the one or more parameters for the coverage can include at least one of duration, intended use case, expected activity load, or geographic region of operation. In some implementations, the system can transmit the digitally verifiable policy credential to each of the plurality of systems. In some implementations, each of the plurality of requests to provide the digitally verifiable policy credential can include criteria for use of the policy. In some implementations, the system can cause the entity providing the AI agent to present a graphical interface representing at least a subset of the plurality of requests. In some implementations, the request can be an application programming interface (API) request, and the system can transmit an API response to the API request that includes instructions to present at least some of the plurality of requests at a graphical user interface (GUI) of at least one user device associated with the entity. In some implementations, the digitally verifiable policy credential can be a machine-readable data object that includes at least one of a first identifier corresponding to the policy, criteria for use of the policy, a second identifier corresponding to a system of the plurality of systems associated with the selected request, an identifier of the entity providing the AI agent, or the deployed agent fingerprint for the AI agent.

At least one other aspect relates to a method. The method can be performed, for example, by one or more processors coupled to non-transitory memory. The method can include receiving through an exchange from an entity providing an AI agent a request for the AI agent to obtain a digitally verifiable policy credential for coverage of the AI agent, the request including a deployed agent fingerprint for the AI agent, at least one of a digitally signed verifiable credential of the AI agent or a digitally signed verifiable license of the AI agent, and one or more parameters for the coverage. The method can include cryptographically validating the at least one of the digitally signed verifiable credential using a public key of a credentialing entity or the digitally signed verifiable license using a public key of a licensing entity. The method can include determining that a hash of the deployed agent fingerprint matches a hash of at least one of the digitally signed verifiable credential or the digitally signed verifiable license. The method can include providing, to a plurality of systems corresponding to policy credentialing entities, an agent data package that includes the deployed agent fingerprint, the at least one of the digitally signed verifiable credential or the digitally signed verifiable license, and the one or more parameters for the coverage. The method can include receiving, from the plurality of systems, a plurality of requests to provide the digitally verifiable policy credential. The method can include receiving a selection of one of the plurality of requests. The method can include generating the digitally verifiable policy credential according to the selected request, the digitally verifiable policy credential including the deployed agent fingerprint, a policy corresponding to the one or more parameters, and the digitally verifiable policy credential digitally signed based on a key of the exchange. The method can include transmitting the digitally verifiable policy credential to the entity.

In some implementations, the method can include providing instructions to the entity providing the AI agent to store at least part of the agent data package in a compliance registry. In some implementations, the method can include providing instructions for at least one of the plurality of systems to store the agent data package in a database. In some implementations, the method can include assigning the AI agent to a class of a plurality of classes, the plurality of classes corresponding to levels of incident probability, based on at least one of the digitally signed verifiable credential or the digitally signed verifiable license of the AI agent. In some implementations, the one or more parameters for the coverage can include at least one of duration, intended use case, expected activity load, or geographic region of operation. In some implementations, the method can include transmitting the digitally verifiable policy credential to each of the plurality of systems. In some implementations, the method can include providing the digitally verifiable policy credential with criteria for use of the policy. In some implementations, the method can include causing the entity providing the AI agent to present a graphical interface representing at least a subset of the plurality of requests. In some implementations, the request can be an application programming interface (API) request, and the method can include generating an API response to the API request that includes instructions to present at least some of the plurality of requests at a graphical user interface (GUI) of at least one user device associated with the entity.

At least one aspect relates to a system. The system can receive, through an exchange from an entity providing an AI agent, a request for the AI agent to obtain a digitally verifiable policy credential for coverage of the AI agent. The request can include a deployed agent fingerprint for the AI agent, at least one of a digitally signed verifiable credential of the AI agent or a digitally signed verifiable license of the AI agent, and one or more parameters for the coverage. The system can cryptographically validate the at least one of the digitally signed verifiable credential using a public key of a credentialing entity or the digitally signed verifiable license using a public key of a licensing entity. The system can determine that a hash of the deployed agent fingerprint matches a hash of at least one of the digitally signed verifiable credential or the digitally signed verifiable license. The system can provide, to a plurality of systems corresponding to policy credentialing entities, an agent data package that includes the deployed agent fingerprint, the at least one of the digitally signed verifiable credential or the digitally signed verifiable license, and the one or more parameters for the coverage. The system can receive, from the plurality of systems, a plurality of requests to provide the digitally verifiable policy credential. The system can generate the digitally verifiable policy credential according to at least one of the plurality of requests, the digitally verifiable policy credential including the deployed agent fingerprint, a policy corresponding to the one or more parameters, and the digitally verifiable policy credential digitally signed based on a key of the exchange.

These and other aspects and implementations are discussed in detail below. The foregoing information and the following detailed description include illustrative examples of various aspects and implementations and provide an overview or framework for understanding the nature and character of the claimed aspects and implementations. The drawings provide illustration and a further understanding of the various aspects and implementations and are incorporated in and constitute a part of this specification. Aspects can be combined, and it will be readily appreciated that features described in the context of one aspect of the invention can be combined with other aspects. Aspects can be implemented in any convenient form, for example, by appropriate computer programs, which may be carried on appropriate carrier media (computer readable media), which may be tangible carrier media (e.g., disks) or intangible carrier media (e.g., communications signals). Aspects may also be implemented using any suitable apparatus, which may take the form of programmable computers running computer programs arranged to implement the aspect. As used in the specification and in the claims, the singular form of ‘a,’ ‘an,’ and ‘the’ include plural referents unless the context clearly dictates otherwise.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIG. 1 is a block diagram of a system to generate an agent fingerprint, in accordance with one or more implementations;

FIG. 2 is a flow chart illustrating a method for generating an agent fingerprint, in accordance with one or more implementations;

FIG. 3 is a block diagram of an example of system to generate a deployed agent fingerprint, in accordance with one or more implementations;

FIG. 4 is a flowchart illustrating a method for generating and validating a deployment fingerprint for an AI agent, in accordance with one or more implementations;

FIG. 5 is a block diagram illustrating an example of a system to deploy AI agents, in accordance with one or more implementations;

FIG. 6 is a flow chart illustrating a method for deploying an AI agent, in accordance with one or more implementations;

FIG. 7 is a block diagram illustrating an example of a system to credential AI agents, in accordance with one or more implementations;

FIG. 8 is a flow chart illustrating a method for credentialing an AI agent, in accordance with one or more implementations;

FIG. 9 is a block diagram illustrating an example of a system to validate AI agents, such as for licensing of AI agents, in accordance with one or more implementations; and

FIG. 10 is a flow diagram illustrating a method for validating an AI agent, in accordance with one or more implementations.

FIG. 11 is a block diagram of an environment for AI agent credentialing;

FIG. 12 is a flow chart illustrating a method for AI agent credentialing; and

FIG. 13 is a flow diagram illustrating a process for AI agent credentialing.

### DETAILED DESCRIPTION

Below are detailed descriptions of various concepts related to, and approaches, methods, apparatuses, and one or more processors for implementing the various techniques described herein. The various concepts introduced above and discussed in greater detail below may be implemented in any of numerous ways, as the described concepts are not limited to any particular manner of implementation. Examples of specific implementations and applications are provided primarily for illustrative purposes.

The techniques described herein relate to systems and methods for technical verification and/or credentialing of AI agents. AI agents can operate in a variety of deployment infrastructures, including systems where models, data assets, instruction sets, and executable containers are managed across multiple infrastructures. The AI agents can perform computational tasks for domains such as healthcare, finance, manufacturing, or legal services. The AI agent can include a combination of modular components that define its behavior, including, for example and without limitation, a trained model, control logic, and external service integrations. The configuration and deployment of such agents can occur across diverse systems, often through automated orchestration tools or continuous integration pipelines that assemble the components of the agent from declarative definitions.

Existing approaches for identifying or validating running AI systems can face limitations in verifying that an operational instance of an AI agent actually matches a declaration configuration for the AI agent, such as a list of components or version information for the AI agent. Conventional techniques can authenticate data transactions or static files but fail to provide a reproducible linkage between the identity of an AI system and the particular collection of software, data, and dependencies that define its function at deployment time. Without such linkage, performance criteria for the AI agent, such as reproducibility or accuracy in generating expected outputs may not be possible to evaluate.

Conventional approaches for deploying AI agents often rely on data that is descriptive but not cryptographically verifiable. Build systems or orchestrators can record version identifiers and configuration files, but such records can be mutable and may not establish a deterministic proof of the deployed artifact. When multiple instances of an agent share common containers or datasets, small divergences in versions or weakly tracked parameters can lead to configuration drift. This drift can cause inconsistencies between declared and operational states of the agent. Furthermore, the absence of strong linkage between deployment metadata and the underlying components can limit the ability of external parties to validate the integrity of a deployed agent without full disclosure of proprietary assets.

The techniques described herein can provide a verifiable framework in which an artificial intelligence agent can be associated with an immutable, cryptographically derived identifier referred to as an agent fingerprint. The agent fingerprint can represent a composite hash derived from individually hashed component manifests, such that the complete identity of an agent can be recovered from its constituent parts. A second derivation referred to as a deployed agent fingerprint can extend the base fingerprint by incorporating deployment-specific parameters, user-defined configurations, and environmental bindings. The two-tier structure can provide a hierarchical linkage between base models produced by a manufacturer and field-deployed instances operated by downstream systems (e.g., as managed by customers or partners). The system can use the agent fingerprint and/or the deployed agent fingerprint as both a cryptographically verifiable identifier for the AI agent and a blueprint or configuration that can be used to execute deployment of the AI agent.

In some examples, the techniques can generate the agent fingerprint by applying a cryptographic hash function to manifests that describe the agent's data sources, model definitions, instruction sets, and runtime software packages. The resulting component identifiers can be aggregated into a metadata file that defines the agent structure. The metadata file can then be canonicalized and hashed to compute a deterministic agent fingerprint. A similar process can be applied when producing a deployed agent fingerprint, where the system can incorporate customer data, deployment configurations, and dependency declarations. The same metadata files can also be used by deployment tools that instantiate the agent through automated workflows, thereby maintaining a cryptographically consistent link between the defined configuration and the physical execution environment. The system can use the agent fingerprint to trigger a configuration-driven deployment approach, which can ensure that any change to an agent's metadata file directly defines and automates its deployment process, enabling continuous integration and delivery with deterministic reproducibility across environments.

For example, the system can generate the agent fingerprint and/or deployed agent fingerprint to facilitate transparency and/or provenance, such as to provide an immutable bill of materials for any given AI agent. The system can provide a technical solution for trust and/or confidence in the AI agent, such as to enable rigorous and/or independent credentialing and licensing. The system can facilitate traceability and/or auditability, as each action executed by a deployed agent can be traced back to the deployed agent fingerprint, which can serve as the unique, verifiable identity for the deployed agent, which can create a clear audit trail. The system can facilitate accountability and/or liability; for example, by uniquely identifying an agent (as well as the provenance of its underlying components), liability for its actions can be more clearly assessed and risk adjusted, which can facilitate a technical implementation for a functional insurance market. The system can provide for greater reproducibility, as the fingerprinting mechanism can ensure that a certified agent's behavior can be precisely reproduced for testing, validation, and/or incident investigation. The system can allow for technical implementation of safety and/or recall functionality across deployments of AI agents, as the ability to uniquely identify every agent instance in the field can allow for targeted recalls or deactivations if a flaw or vulnerability is discovered.

The techniques described herein can provide a reliable method for maintaining integrity, traceability, and/or reproducibility of AI agents throughout their lifecycle. By deriving fingerprints directly from canonicalized component data, the techniques eliminate inconsistencies introduced by environmental or human factors. Validation entities can verify that a running agent matches its declared configuration by recomputing the fingerprints from observed components. Deployment orchestrators can use the same metadata to instantiate consistent environments without manual intervention. Through these technical capabilities, the approaches described herein can establish a deterministic link between identity, configuration, and execution for AI agents.

The techniques described herein can improve the reliability and verifiability of distributed AI deployments by enabling objective testing of AI agents against defined evaluation criteria. By anchoring each deployment to a deterministic fingerprint, the disclosed methods can ensure that an AI agent subjected to testing can be reliably identified and verified as the intended version under evaluation. These mechanisms can reduce ambiguity caused by configuration drift and mutable versioning information, allowing independent verification systems to test the agent's responses against selected benchmarks or rubrics. Through these verifiable linkages, credentialing, licensing, or auditing processes can confirm that a running agent not only originates from the proper configuration but also demonstrates measurable performance under test conditions. The techniques can thereby strengthen data provenance, reproducibility, and accountability in verifying the operational behavior of artificial intelligence agents across cloud, edge, and on-premises environments.

For example, to address the technical challenges in verifying credentials for AI agents, systems and methods in accordance with the present disclosure can generate and/or verify a deterministic cryptographic fingerprint that uniquely represents the composition of an AI agent and use that fingerprint to validate test results. The fingerprint can encapsulate the various components that define an agent, such as model parameters, instruction files, datasets, software builds, and approved external dependencies, ensuring that the correct version of the agent is subjected to evaluation. By establishing a secure, verifiable relationship between an agent's declarative metadata, its operational instantiation, and the testing process, the techniques can allow independent entities (e.g., credential granting organizations) to confidently assess whether a deployed agent meets the specific performance or behavioral criteria defined by the associated tests.

The techniques described herein can yield several technical improvements. They can provide a deterministic mechanism for verifying that a deployed AI agent both matches its declared configuration and satisfies the prescribed evaluation standards during testing. The use of cryptographic hashing of component manifests can create immutable identifiers that support reproducibility when retesting agents in distributed environments. The linkage between metadata-driven configuration, deployment, and test execution can prevent configuration drift and ensure that performance measurements are made against an authenticated and reproducible system state. As a result, the disclosed techniques can establish a transparent provenance and validation chain for AI agents, providing a measurable foundation for trust, reproducibility, and regulatory compliance in environments that depend on objective testing for automated decision-making systems.

Systems and methods in accordance with the present disclosure can validating AI agents for licensing in digital environments in accordance with operational constraints, such as performance criteria, constraints on types of outputs generated, or constraints on actions that the AI agents can perform autonomously or may be required to present to an authorized user for authorization. For example, AI agents can perform automated reasoning, data transformation, or decision-making tasks within computing systems that interact with both automated and human-operated components. The agents can include computational models, executable instructions, and data assets that collectively generate requested outputs. Licensing processes can allow such agents to operate within defined scopes, such as professional or regulatory domains, so that system operators and authorities can rely on predictable and verifiable agent behavior. In such contexts, validation of the technical identity and operating configuration of each agent instance can provide an evidentiary basis for granting or regulating authorization to deploy those agents.

Conventional licensing workflows for AI agents generally rely on static credentials or descriptive attestations that do not confirm whether a deployed instance corresponds to its approved configuration. For example, after initial evaluation, an artificial intelligence agent may alter software components, model parameters, or external data references, which can cause the deployed configuration to diverge from the configuration that underwent prior assessment. Traditional verification methods do not cryptographically confirm that the executing agent matches the certified composition, nor do such methods provide real-time validation of prerequisite credentials associated with a target license. As a result, conventional systems cannot reliably verify agent authenticity or continued conformity to licensing prerequisites during field operation.

The techniques described herein can provide validation processes for licensing AI agents using cryptographically verifiable evidence. The approach can combine a deployed agent fingerprint, a verifiable credential, and a verifiable license to form an interconnected set of artifacts that link agent identity, credential authorization, and active licensing status. A licensing system can receive a request containing the fingerprint and credential, use public key validation to authenticate the credential, and confirm that the fingerprint reflects the verified agent configuration. The system can then determine that the credential meets defined prerequisites for a given license and can issue a digitally signed, verifiable license that encodes hashes, scope conditions, and provenance information. By correlating agent identity and credential evidence through cryptographic validation, these techniques can maintain continuous assurance that licensed artificial intelligence agents operate within approved jurisdictional and functional boundaries.

The techniques described herein relate to digital credentialing and verification of AI agents operating, for example, in regulated environments, such as to authorize deployment of the AI agents based on policies that include coverage terms designed to mitigate potential losses associated with performance deviations. For example, the AI agents may be deployed across sectors such as healthcare, financial services, or legal services. In these sectors, authorization for deployment may depend on credentials, licenses, and/or policy coverage associated with a given AI agent. Conventional credentialing and policy management approaches often rely on static databases, which can be susceptible to invalid or inauthentic data due to the absence of robust version control and authenticity verification mechanisms. For example, such approaches may be vulnerable to discrepancies between credentials and model versions following deployment configuration changes, as well as to acceptance of inauthentic credentials due to insufficient verification mechanisms. The techniques described herein provide a unified, cryptographically verifiable framework in which deterministic fingerprints uniquely identify AI agent deployments and serve as a common reference point for digitally signed credentials, licenses, and policy records issued by credentialing entities, licensing authorities, and policy providers. A credential exchange system can validate submitted artifacts through cryptographic signature verification and hash matching, distribute verified agent data to multiple participating systems, and enable issuance, aggregation, and verification of digitally signed policy offers that reference the same agent fingerprint. As a result, the credential exchange system can verify that the credentials match the deployment configuration (e.g., version) of the given AI agent and are authentic. The resulting digitally verifiable policy credential can therefore bind selected coverage terms to the specific AI agent deployment, establishing a continuous provenance chain that supports automated authentication, real-time compliance evaluation, transparent risk assessment, and improved trust and accountability across distributed systems employing AI agents.

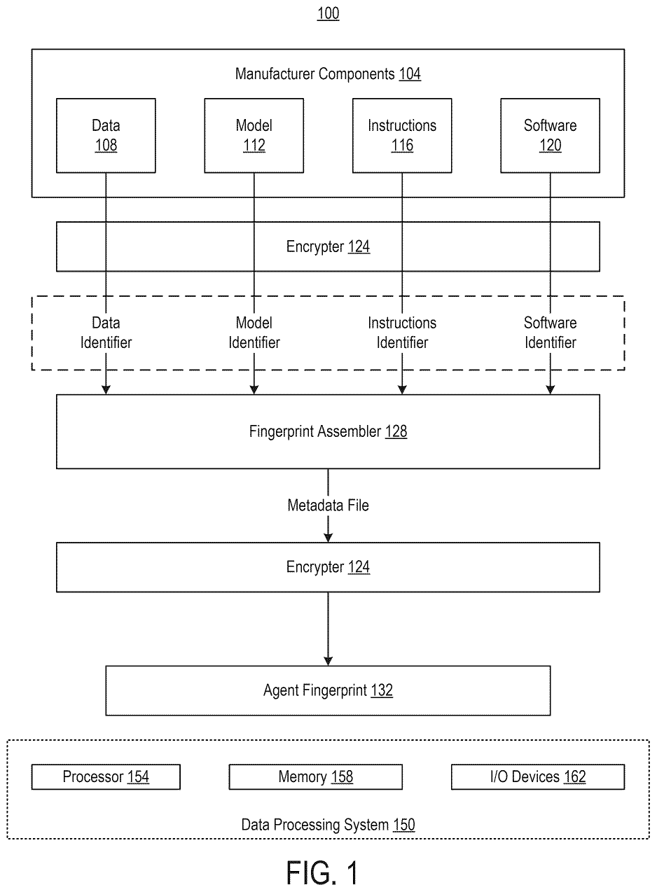

Referring now to FIG. 1, in brief overview, illustrated is a block diagram of a system 100, such as an agent fingerprint generation system 100. The system 100 can generate a deterministic identifier of an AI agent, derived from manufacturer components of the AI agent. For example, the system 100 can include manufacturer components 104, which can include data 108, model 112, instructions 116, and software 120. The system 100 can further include an encrypter 124 and a fingerprint assembler 128, and can output an agent fingerprint 132. The system 100 can include or be implemented using one or more data processing systems 150.

Referring to FIG. 1 in further detail, the system 100 can be or include a computing platform (e.g., data processing system 150) that processes component data of an AI agent to produce a deterministic fingerprint representing the AI agent. For example, the system 100 can be implemented as at least one of a cloud-based build pipeline or an on-premises system including one or more CPUs or GPUs. The system 100 can obtain component manifests, can generate hashes, and can assemble these into metadata files used to define an agent's identity. As an example, the system 100 can compute cryptographic digests for datasets, models, instruction files, and container images, which the system 100 can combine into a canonical metadata structure. The system 100 can perform deterministic hashing and metadata assembly procedures to compute a reproducible agent fingerprint based on canonical ordering rules. For example, the system 100 can use encryption, e.g., SHA-256 encryption, and can apply JSON canonicalization, to avoid environmental variance across builds. As depicted in FIG. 1, the data processing system 150 can include one or more processors 154, one or more memory (e.g., memory devices) 158, and/or one or more input/output (I/O) devices 162. The processor 154 can be a general purpose or specific purpose processor, an application specific integrated circuit (ASIC), one or more field programmable gate arrays (FPGAs), a group of processing components, or other suitable processing components. The processor 154 may be configured to execute computer code or instructions stored in memory (e.g., fuzzy logic, etc.) or received from other computer readable media (e.g., CDROM, network storage, a remote server, etc.) to perform one or more of the processes described herein. The memory 158 may include one or more data storage devices (e.g., memory units, memory devices, computer-readable storage media, etc.) configured to store data, computer code, executable instructions, or other forms of computer-readable information. The memory 158 may include random access memory (RAM), read-only memory (ROM), hard drive storage, temporary storage, non-volatile memory, flash memory, optical memory, or any other suitable memory for storing software objects and/or computer instructions. The processor 154 can be implemented as a hardware processor including a Central Processing Unit (CPU), an Application-Specific Integrated Circuit (ASIC), an Application-Specific Instruction-Set Processor (ASIP), a Graphics Processing Unit (GPU), a Physics Processing Unit (PPU), a Digital Signal Processor (DSP), a Field Programmable Gate Array (FPGA), a Programmable Logic Device (PLD), a Controller, a Microcontroller unit, a Processor, a Microprocessor, an ARM, or the like, or any combination thereof. The memory 158 may include database components, object code components, script components, or any other type of information structure for supporting the various activities and information structures described in the present disclosure. The memory 158 can include various modules (e.g., circuits, engines) for completing processes described herein. The I/O devices 162 can include any one or more communications electronics (e.g., wired or wireless reception and/or transmission circuitry; communications busses; etc.), and can include any one or more user interface devices (e.g., displays, microphones, keyboards, mouse devices, touch input devices, etc.) to facilitate communication with the data processing system 150 and/or one or more components thereof. The data processing system 150 can be implemented in any of various computing platforms or architectures, including but not limited to any of various client-server architectures.

As illustrated in FIG. 1, the system 100 can include or obtain one or more components 104, such as manufacturer components 104 that a manufacturer of an AI agent provides to facilitate execution or deployment of the AI agent. The components 104 can be or include any one or more data structures, software, firmware, code, scripts, or pointers or identifiers thereof. The manufacturer can include an entity that develops, stores, or provides the components 104, e.g., via one or more repositories. For example, the manufacturer can be a manufacturer of record of the AI agent. The AI agent can include or be defined according to one or more of the components 104.

The AI agent can include one or more neural networks, language models, multimodal models, or combinations thereof, such as represented by model 112 of the components 104. The AI agent can be a self-contained computational entity that performs one or more tasks autonomously by processing data and executing algorithms that emulate human reasoning, decision-making, or problem-solving, for example and without limitation. The AI agent can include multiple interdependent components such as a trained model (e.g., trained model 112), data inputs (e.g., data 108), instructions (e.g., prompts and/or instructions 116), and/or executable software (e.g., software 120), which can determine or manage the operation or behavior of the AI agent. In some implementations, the AI agent can operate as a composite system integrating neural network architectures, reinforcement learning modules, and rule-based logic to generate context-dependent outputs. For example, a language-processing AI agent can receive textual inputs, can tokenize the inputs into interpretable elements, and can produce coherent responses derived from a parameterized transformer model. In some implementations, the AI agent can interact with external systems and data stores, can apply learned policies to dynamic environments, and can (continuously) update its internal state variables to optimize performance metrics. The AI agent can process numeric, categorical, linguistic, or multimodal data and can execute within a distributed infrastructure that includes cloud-based servers, local computing nodes, or edge devices. Each AI agent can maintain an internal configuration defining the parameters, data references, and operational contexts under which it performs assigned tasks, allowing deterministic instantiations for analysis, testing, and deployment across technical environments.

Referring further to FIG. 1, the components 104 can be elements (e.g., provided by the manufacturer) of a base definition of the AI agent. The base definition can represent, for example, a minimum or sufficient set of components 104 to allow for functionality of the AI agent (e.g., even if additional user data or other resources may be expected to be used to facilitate deployment of the AI agent according to one or more criteria of a user). For example, the components 104 may represent data files, trained models, instruction sets, and/or software containers, such as may be generated under controlled build conditions. The manufacturer components 104 can serve as the inputs from which individual component identifiers are computed for inclusion in the agent fingerprint, as described further herein.

The components 104 can include data 108. The data 108 can include any of various datasets, data manifests, or data assets that the AI agent (e.g., model 112) uses for training (including any of various unsupervised learning, supervised learning, fine-tuning, transfer learning, or in context-learning) and/or inference (including, for example and without limitation, for context data that the model 112 refers to in order to perform inference, or for retrieval, such as for retrieval-augmented generation (RAG)). For example, the data 108 may include structured medical datasets, language corpora, or tabular data used for specific domain adaptation. The data 108 can include any of various text, speech, audio, image, and/or video data. The data 108 can include structured or unstructured data. The data 108 can include training data pairs, such as training data elements that are each associated with respective labels.

The components 104 can include at least one model 112. The model 112 can include any one or more functions, algorithms, machine learning models, neural networks, reinforcement learning models, language models, large language models (LLMs), small language models (SLMs), multimodal language models, or combinations thereof. The model 112 can include data representing the structure of the model 112, such as any one or more configurations of the model 112, such as weights, biases, parameters, versions, base models, architectures, tuning parameters, arrangements or types of network layers, connections between layers, types of input data or output heads, or various combinations thereof. For example, the model 112 can represent a trained neural network or statistical model implementing an inferencing capability of the AI agent. For example, the model 112 may correspond to a large language model, a fine-tuned transformer, or other parametric architecture.

In some implementations, the components 104 include instructions 116. The instructions 116 can include at least one of instructions or prompts that the model 112 can process to perform corresponding actions. In some implementations, the model 112 uses at least a portion of instructions 116 as context. In some implementations, the model 112 can combine (e.g., append) one or more instructions 116 to prompts received from a user or other system to input to the model 112, e.g., to input to the AI agent The instructions 116 can represent operational control data or prompt templates directing behavior of the AI agent. For example, instructions 116 may be files including initialization prompts or system configurations guiding contextual responses.

In some implementations, the components 104 can include software 120. The software 120 can include or be coupled with or reference any one or more code, scripts or firmware, for example, that can execute the AI agent, such as to provide at least one of an application layer or an interface for or to the AI agent. For example, the software 120 can include executable code or a containerized image that packages runtime dependencies for the AI agent. For example, the software 120 can include container image digests that encapsulate environment variables, dependency libraries, and operating system layer hashes.

The agent fingerprint generation system 100 can include an encrypter 124. The encrypter 124 can include any one or more code, scripts, software, algorithms, functions, rules, or combinations thereof to perform operations such as applying an encryption, such as a cryptographic operation, on components 104. For example, the encrypter 124 can be a cryptographic engine, and can computes one or more hashes (e.g., hash values) of data inputted to the encrypter 124, such as to compute hashes of the respective components 104. In some implementations, the encrypter 124 includes a tokenizer or an embedding model. In some implementations, the encrypter 124 can implement a Federal Information Protection Standard (FIPS)-compliant algorithm to perform the encryption. The encrypter 124 can apply a cryptographic operation, such as SHA-256 or another deterministic hash function, to the components 104. For example, as described further herein, the encrypter 124 can receive a component 104 (e.g., any of the data 108, model 112, instructions 116, and/or software 120), and compute a hash (e.g., a cryptographic hash) of the component 104 to generate a corresponding identifier of the component (e.g., data identifier of the data 108; model identifier of the model 112; instructions identifier of the instructions 116, software identifier of the software 120). The encrypter 124 can generate the hashes to be encoded or encrypted representations of respective components 104, such as machine-readable representations.

In some implementations, the encrypter 124 hashes each of the components 104 to produce the identifiers as unique identifiers (which collectively can form the metadata record and/or metadata file). The encrypter 124 can ingest the components 104 for cryptographic derivation of the identifiers. As an example, the encrypter 124 can pass each file or manifest of the components 104 into an SHA-256 hashing process, to generate the respective identifier.

For example, the encrypter 124 can hash the data 108 to generate the data identifier. The data identifier can be a digest that represents a canonical form of the data 108. For example, the encrypter 124 may receive a manifest describing source repositories or data revisions, and can output a single cryptographic hash as the data identifier. The data identifier can be used by a deployment system to mount a correct, versioned dataset for the AI agent.

The encrypter 124 can hash the model 112 to generate the model identifier. The model identifier can convey the version and parameter configuration used in the build of the model 112. For example, the encrypter 124 can hash the model 112 to include the model architecture file and the configuration parameters of model 112 in a manifest represented by the model identifier, such as where a checksum of the manifest forms the model identifier. The model 112 may be input to the encrypter 124, which can compute the model identifier as a cryptographic hash of the model 112. The model identifier can be used by a deployment system to load the correct model files.

The encrypter 124 can hash the instructions 116 to generate the instructions identifier. The instructions identifier can be a unique identifier describing the instructions 116, such as where the instructions 116 include a version-controlled instruction set. For instance, the instructions identifier can be mapped to a version-controlled file (e.g., via Git commit hash) that is loaded by a deployment system at runtime.

The encrypter 124 can hash the software 120 to generate the software identifier. The software identifier can specify an exact executable environment for deployment of the AI agent. For example, the software identifier represent a specific and/or pullable container image digest (e.g., sha256: . . . ) that an orchestrator, such as Kubernetes, can used to deploy the exact software build for the AI agent. For example, the software identifier can be used as a direct and/or executable pointer.

Referring further to FIG. 1, the system 100 can include a fingerprint assembler 128. The fingerprint assembler 128 can include any one or more functions, algorithms, rules, policies, heuristics, models, or combinations thereof to perform operations such as to generate a data structure, such as a metadata file, based at least on the identifiers of the components 104 that the encrypter 124 generates. For example, the fingerprint assembler 128 can generate the metadata file according to a structure for the metadata file, such as an order of inclusion of data in fields of the metadata file. For example and without limitation, the structure can represent a set of name-value pairs, ordered lists, and/or comma-separated values. The fingerprint assembler 128 can generate the metadata file as a JSON file.

In some implementations, the fingerprint assembler 128 generates the metadata file to include an identifier of the manufacturer of the AI agent. For example, the fingerprint assembler 128 can use the encrypter 124 to hash a name or other identifier of the manufacturer to generate the identifier of the manufacturer. The fingerprint assembler 128 can generate the metadata file to include an identifier of the AI agent, such as a name or version of the AI agent; the identifier of the AI agent may be human-readable (or can be hashed to be a machine-readable identifier).

The fingerprint assembler 128 can generate the metadata file to include the identifiers of the components 104 from the encrypter 124. For example, the fingerprint assembler 128 can assemble the data identifier, model identifier, instructions identifier, and software identifier into the structure of the metadata file. By referencing the identifiers, the metadata file can allow for reproducible access to the components 104 (e.g., referencing the data identifier can allow for reproducible access to the datasets of the data 108 to use for operation of the AI agent).

The fingerprint assembler 128 can combine the identifiers into the structure of the metadata file, which can be used for agent validation and future deployments. The fingerprint assembler 128 can generate the metadata file to include data for one or more fields of the identifier of the manufacturer, the identifier of the AI agent, a timestamp indicating when the AI agent build was finalized, the identifiers of the components 104, and/or external dependency manifest data. The fingerprint assembler 128 can perform canonical ordering (e.g., according to a metadata schema) of the name-value pairs of such fields to prepare the structure for cryptographic hashing. As an example, the fingerprint assembler 128 can perform key ordering and array normalization steps to avoid nondeterministic serialization. In some implementations, the fingerprint assembler 128 generates the metadata file to include a version string for the metadata schema, which can further facilitate reliable use of the metadata file.

In some implementations, the fingerprint assembler 128 generates the metadata file to include (one or more identifiers of) one or more manifests of external dependencies for the AI agent. This can include, for example, an array declaring the types of external services the AI agent is architected to use, acting as a manifest of approved tool slots. The manifest of external dependencies can be used by a deployment system to pre-configure network access or service bindings, for example.

In some implementations, the fingerprint assembler 128 generates the metadata file to include (one or more identifiers of) an interface specification for the AI agent. For example, the fingerprint assembler 128 can include an identifier, such as a hash, of the interface specification (e.g., to a remote system, such as a model provider) that the dependency is to conform to, such as for automated client generation or interface testing.

The following is an illustrative example of a metadata file, including the identifiers and the schema for the metadata file:
{
“format_version”: “1.1”,
“manufacturer_id”: “87b1c428-2c67-428a-a195-21a4c4202c2e”,
“agent_model_name”: “MediBot-Nurse-v3.2-Intake”,
“timestamp”: “2025-08-27T10:00:00Z”,
“components”: {
“data_cid”:
“sha256:a1b2c3d4e5f678901234567890abcdef1234567890abcdef1234567890ab”,
“model_cid”:
“sha256:b2c3d4e5f6a1234567890abcdef1234567890abcdef1234567890abcde”,
“instructions_cid”:
“sha256:c3d4e5f6a1b234567890abcdef1234567890abcdef1234567890abcd”,
“software_cid”:
“sha256:d4e5f6a1b2c34567890abcdef1234567890abcdef1234567890abcde”
},
“external_dependencies_manifest”: [
{
“dependency_id”: “EMBEDDINGS_PROVIDER”,
“description”: “Service for generating text embeddings for RAG.”,
“interface_spec_cid”: “sha256:2b3c4d5e6f7a8901234567890abcdef1234567890abcdef1234567890”
},
{
“dependency_id”: “PATIENT_LOOKUP_API”,
“description”: “Tool for retrieving patient records from an EMR.”,
“interface_spec_cid”: “sha256:3c4d5e6f7a2b901234567890abcdef1234567890abcdef123456789”
}
]
}

Referring further to FIG. 1, the encrypter 124 (which can be a same encrypter 124 that encrypts the components 104 into respective data, model, instructions, and/or software identifiers, or a different encrypter 124 or instance of an encrypter 124) can encrypt the metadata file to generate an agent fingerprint 132. For example, the encrypter 124 can hash the metadata file (e.g. and without limitation, using SHA256) to generate the agent fingerprint 132. The agent fingerprint 132 can be a cryptographic representation of the metadata file, such as to allow for a compact and/or verifiable representation of the AI agent and the components 104 used to deploy the AI agent.

The agent fingerprint 132 can represent the unique, deterministic identifier of the AI agent as produced by the manufacturer. For example, the agent fingerprint 132 can be the SHA-256 digest of the canonicalized AF metadata file describing all core components. The agent fingerprint 132 can be used as a verifiable reference or blueprint to validate any instance of the AI agent during deployment or credentialing. The system 100 can store the agent fingerprint 132 may be stored in a registry or deployment system for retrieval during later validation or licensing workflows. As an example, the system 100 can apply the agent fingerprint as a metadata tag, e.g., an immutable metadata tag, to a container image for the AI agent. This can allow the system 100 to facilitate verifiable use of the AI agent upon retrieval of the container image.

Referring further to FIG. 1, the system 100, e.g., using the encrypter 124, can generate component identifiers from manufacturer component inputs, can assemble the component identifiers (along with any of various other identifiers as noted above) into the structure of the metadata file, and can hash the assembled metadata file to form the agent fingerprint 132. For instance, the system 100 can first compute per-component identifiers, and can subsequently execute a final hash pass over the canonicalized JSON object produced by the fingerprint assembler 128. The encrypter 124 may perform these operations by serializing component manifests into a canonical format before computing digest outputs. As an example, canonicalization may include alphabetically ordering keys and removing extraneous whitespace prior to the hash computation.

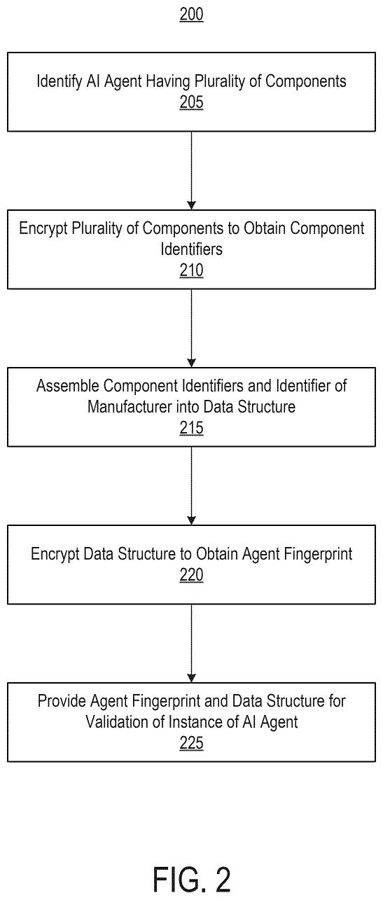

Referring now to FIG. 2, illustrated is a flow chart of a method 200 for generating an agent fingerprint for an AI agent. The method 200 can be executed, performed, or otherwise carried out by any of various systems described herein, including one or more components of the system 100. In brief overview of the method 200, the method 200 can include identifying an AI agent having a plurality of components 205, encrypting the plurality of components to obtain component identifiers 210, assembling the component identifiers and a manufacturer identifier into a data structure 215, encrypting the data structure to obtain an agent fingerprint 220, and providing the agent fingerprint and the data structure for validation of an instance of the AI agent 225. The method 200 or one or more operations of the method 200 can be triggered responsive to any of a variety of events, such as a request for validation or deployment of the AI agent, or in response to detection of a change in one or more components (e.g., components 104) of the AI agent.

At 205, the method 200 can include identifying an AI agent that includes or is associated with a plurality of components. For example, one or more manifests or repositories that define the constituent components of the AI agent can be accessed. Atomic elements for the AI agent such as data files, model parameters, instruction manifests, and/or executable software components can be identified, each representing a discrete element of the agent's operational configuration. In some implementations, version-controlled directories associated with a build environment of the AI agent can be processed to obtain resource descriptors that collectively define an operational scope for the agent. For example, the agent fingerprint generation system can enumerate structured references identifying model artifacts, data resources used for retrieval-augmented generation (RAG), container image references, and interface dependency manifests stored in respective repositories. The identification can occur at build initialization or when a manufacturer prepares a release candidate for fingerprint generation, such as during an automated continuous integration or continuous deployment pipeline that executes after source repositories containing model, data, and configuration definitions are checked out. The identification can occur responsive to a request to deploy or validate the AI agent.

At 210, the method 200 can include encrypting the plurality of components to obtain component identifiers of the plurality of components. Each component of the AI agent can be processed (e.g., encrypted, encoded) to generate a deterministic hash value, which can uniquely represent the data or content of the respective component content. In some implementations, a FIPS compliant cryptographic function such as Secure Hash Algorithm 256 (SHA-256) can be applied to each retrieved manifest for each component, which describes inputs including data, model, instructions, and/or software. For example, a base data manifest, a model configuration file, an instruction definition, and a container manifest can be read, serialized into a canonicalized form, and subjected to computation of corresponding cryptographic digests, thereby producing a set of component identifiers. In some implementations, canonicalization can involve ordering keys alphabetically, normalizing character encoding, and eliminating redundant whitespace to ensure that identical manifests always yield the same digest output. For example, a JSON metadata object representing a model configuration can be normalized prior to execution of the SHA-256 algorithm, which can produce a reproducible identifier value that accurately reflects the state of the model inputs within the manufacturing environment.

At 215, the method 200 can include assembling the component identifiers and an identifier of a manufacturer into a data structure, such as a data structure for a metadata file. For example, the identifiers of the components, an identifier of the manufacturer of the AI agent, and/or additional metadata such as timestamps or dependency manifests can be combined into a structured data object. A structured object can be generated as a metadata file containing name-value pairs that associate each identifier with a corresponding field defining the component type. In some implementations, identifiers of the data, model, instructions, and/or software for the AI agent can be merged into the metadata file. The assembly operation can be performed automatically after all component identifiers have been generated, thereby consolidating the component data into a complete and deterministic bill of materials. For example, in a continuous integration pipeline, the assembly can be triggered automatically at completion of the final component identifier hashing process to produce a finalized metadata object. The metadata file can be formatted in accordance with predefined canonicalization rules specifying serialization order and syntax consistency. In some implementations, alphabetical ordering of field names can be maintained, and consistent serialization can be applied across records to allow for reliable reproduction of identical cryptographic verification outputs across environments.

At 220, the method 200 can include encrypting the data structure (e.g., the metadata file) to obtain an agent fingerprint. For example, responsive to assembly of the metadata file, a cryptographic hash can be applied to the metadata file to generate the agent fingerprint, which can provide for a deterministic identifier of the AI agent and the components of the AI agent. In some implementations, the agent fingerprint can be expressed as AF=SHA-256(Canonicalize(AF_Metadata_File)), resulting in a reproducible digital signature derived from the canonicalized metadata content. The hashing operation can be performed after the assembly process is finalized, such as prior to the storage or distribution of the generated fingerprint. In some implementations, hashing can be executed during a final artifact packaging sequence within a continuous integration environment before release to a registry. The output of the hash computation can be verified through checksum comparison to detect any divergence between the computed value and expected reference data.

The method 200 can include providing the agent fingerprint and the data structure (e.g., the metadata file) for validation of an instance of the AI agent (225). For example, the agent fingerprint and the corresponding metadata file can be transmitted or otherwise made accessible to validation, credentialing, or licensing entities for reference in subsequent verification processes. In some implementations, the generated data can be uploaded to a credential-granting organization or deposited within an attestation registry that permits access by authorized verification systems. For example, completion of fingerprint generation and archival operations can precede the transmission phase, allowing the information to be used as a reference record for deployment validation or credential assessment. In some implementations, distribution of the fingerprint and metadata can occur during the final build stage to align the release of the agent with the initiation of credential verification procedures. The fingerprint and metadata can be provided through one or more secure interfaces that facilitate retrieval and comparison of encrypted component identifiers. For example, a verification authority can load a corresponding deployed container image, regenerate an associated fingerprint, and confirm alignment with the manufacturer-declared fingerprint to validate authenticity of the agent instance.

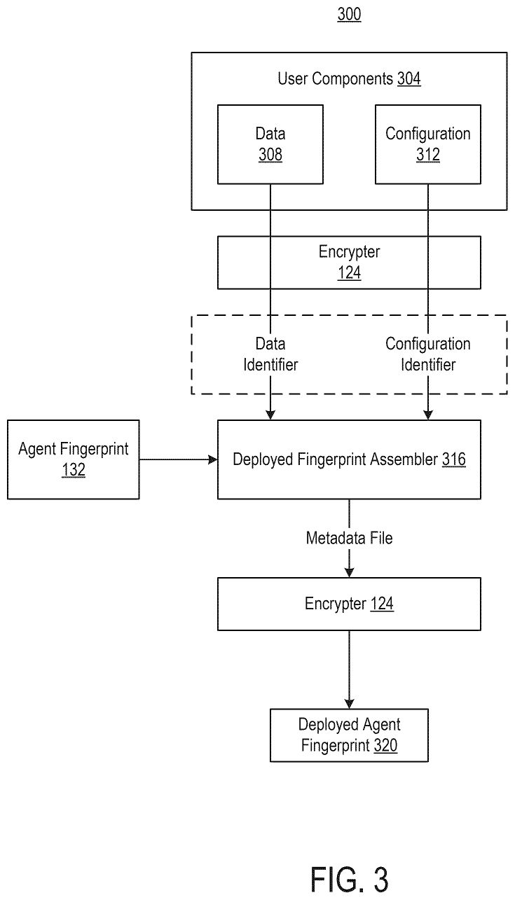

Referring now to FIG. 3, illustrated is a block diagram of a system 300, such as a system for generating a deployed agent fingerprint for a deployed instance of an AI agent. The AI agent can correspond to the AI agent described with reference to FIG. 1, where the deployed instance can be further updated (e.g., customized, modified, trained, fine-tuned, etc.) for a target application, such as for a customer and/or user of the AI agent (e.g., in contrast to the base definition of the AI agent that may be represented by the components 104 and/or agent fingerprint 132). The system 300 can be implemented to generate the deployed agent fingerprint based on customer-specific components and a previously established agent fingerprint. In some implementations, the system 300 obtains the agent fingerprint 132 that the system 100 generates, for example. In brief overview, the system 300 can include one or more user components 304, the encrypter 124, and a deployed fingerprint assembler 316, and can output a deployed agent fingerprint 320. The user components 304 can include data 308 and configuration 312.

As depicted in FIG. 3, the system 300 can include or obtain one or more components 304, which can be user components 304. For example, the components 304 can represent deployment-specific assets supplied by a user, customer, or end organization for use with the AI agent. For example, the user components 304 can include customer-provided data sources, configuration files, and local environment manifests utilized during deployment. The AI agent can use the components 304 to deploy a user-specific (e.g., customer-specific) instance of the AI agent.

The components 304 can include data 308. The data 308 can include one or more datasets or resource manifests for the AI agent. In some implementations, the data 308 includes domain-specific information and/or data for RAG operations that the AI agent is to perform. For example, the data 308 can include medical records datasets, financial policy tables, or other proprietary information local to the deploying organization. The data 308 can include data from or identifiers of any of various data sources.

The user components 304 can include at least one configuration 312. The configuration 312 can include one or more files or manifests that define parameters and/or settings for the deployment of the AI agent. For example, the configuration 312 can specify parameters such as environment variables, model bindings, or resource allocation limits unique to the target environment (e.g., software and/or hardware environment) in which the AI agent is to be deployed.

Referring further to FIG. 3, the system 300 can include the encrypter 124. The encrypter 124 can be configured for the system 300 in a manner analogous to the encrypter 124 of the system 100 (though may be implemented using one or more separate encrypters or encryption functions than used by the system 100). The encrypter 124 can encrypt the components 304 to generate identifiers of the components 304, such as to compute respective hashes of the data 308 and/or configuration 312 to generate a data identifier and/or a configuration identifier. As an example, each asset within the components 304 can be hashed to form identifiers which, as described further herein, the system 300 can append to a metadata file referencing the agent fingerprint.

For example, the encrypter 124 can hash the data 308 form a data identifier, which can represent the dataset to be used in deployment of the AI agent. As an example, the encrypter 124 may compute a SHA-256 digest for a customer data manifest represented by the data 308, which can yield a unique value linked to the corresponding data 308. The data identifier can be a hash of the manifest listing all customer-specific RAG documents or other local data sources. The data identifier can be used by as deployment system to mount the data 308. In some implementations, the encrypter 124 serializes the data 308 before hashing, which can maintain deterministic reproducibility of dataset references. For instance, canonicalization may involve key sorting or format normalization prior to digest computation to ensure consistency across environments.

The encrypter 124 can hash the configuration 312 to generate a configuration identifier, which can represent the customer- (or user) specific settings for deployment of the AI agent. For example, the configuration identifier can be consumed by the deployment system to apply environment variables, feature flags, or resource limits to the deployment of the AI agent. In some implementations, the configuration 312 may be encoded in a canonicalized JSON or YAML structure before processing by the encrypter 124. For instance, the structure can be flattened and normalized before hashing to prevent variations due to whitespace or system differences.

Referring further to FIG. 3, the system 300 can include a deployed fingerprint assembler 316. The deployed fingerprint assembler 316 can be analogous to or include components and/or functionality of the fingerprint assembler 128. The deployed fingerprint assembler 316 can generate a deployment metadata file that includes the agent fingerprint 132 and the hashed identifiers for user-specific components. The deployed fingerprint assembler 316 can perform data processing operations that merge these values into a canonical structure ready for encryption. In some implementations, the deployed fingerprint assembler 316 can construct a JSON object that defines fields such as for the agent fingerprint, the identifier of the data 308, and/or the identifier of the configuration 312. For example, the deployed fingerprint assembler 316 can retrieve the agent fingerprint 132, combine it with the computed identifiers from the encrypter 124, and assemble the combined fields into a structured object. In some implementations, the deployed fingerprint assembler 316 can apply canonicalization routines to establish consistent field ordering across computing environments. For example, the deployed fingerprint assembler 316 can alphabetically sort field names, normalize data types, and validate compliance with a predefined schema before passing the metadata to the encrypter 124 for hashing. The resulting canonical metadata file can be serialized using a deterministic encoding format so that identical logical content produces the same binary representation during subsequent encryption operations.

In some implementations, the deployed fingerprint assembler 316 generates the metadata file to include (one or more identifiers of) one or more deployment dependencies. For example, the identifier(s) can include an array of objects that can indicate an implementation of each corresponding dependency identifier declared in the agent fingerprint. The identifiers of the deployment dependencies can be used by the deployment orchestrator to configure network policies, firewall rules, or service mesh routes, for example and without limitation, such as to ensure that the AI agent can only communicate with its declared dependencies.

The following is an illustrative example of the metadata file generated by the deployed fingerprint assembler 316, including the identifiers and the schema for the metadata file:
{
“format_version”: “1.1”,
“manufacturer_id”: “87b1c428-2c67-428a-a195-21a4c4202c2e”,
“agent_model_name”: “MediBot-Nurse-v3.2-Intake”,
“timestamp”: “2025-08-27T10:00:00Z”,
“components”: {
“data_cid”: “sha256:a1b2c3d4e5f678901234567890abcdef1234567890abcdef1234567890ab”,
“model_cid”: “sha256: b2c3d4e5f6a1234567890abcdef1234567890abcdef1234567890abcde”,
“instructions_cid”: “sha256:c3d4e5f6a1b234567890abcdef1234567890abcdef1234567890abcd”,
“software_cid”: “sha256:d4e5f6a1b2c34567890abcdef1234567890abcdef1234567890abcde”
},
“external_dependencies_manifest”: [
{
“dependency_id”: “EMBEDDINGS_PROVIDER”,
“description”: “Service for generating text embeddings for RAG.”,
“interface_spec_cid”: “sha256:2b3c4d5e6f7a8901234567890abcdef1234567890abcdef1234567890”
},
{
“dependency_id”: “PATIENT_LOOKUP_API”,
“description”: “Tool for retrieving patient records from an EMR.”,
“interface_spec_cid”:
“sha256:3c4d5e6f7a2b901234567890abcdef1234567890abcdef123456789”
}
]
}

Referring further to FIG. 3, the system 300 can use the encrypter 124 to generate a deployed agent fingerprint 320 based on the metadata file (e.g., the metadata file generated by the deployed fingerprint assembler 316). The deployed agent fingerprint 320 can represent a deterministic cryptographic identifier associated with a specific deployment instance of an AI agent that incorporates customer-specific components. In some implementations, the deployed agent fingerprint 320 can be generated by hashing, e.g., applying a cryptographic hash function such as SHA-256, to a canonicalized deployment metadata file representing the deployment-related parameters. For example, the system 300 can compute the fingerprint as SHA-256(Canonicalize(DAF_Metadata_File)), thereby producing a reproducible identifier that establishes a verifiable association between the manufacturer definition and the customer deployment instance. In some implementations, the deployed agent fingerprint 320 can be transmitted to one or more validation systems, which can confirm component alignment and provenance across distinct customer environments. For example, a credential-granting authority can recompute a fingerprint directly from a deployed image and compare its value to the declared deployed agent fingerprint 320 to confirm that the deployment precisely matches the expected configuration. The deployed agent fingerprint 320 can be retained in a version-controlled repository or deployment register to facilitate subsequent verification or attestation of a specific release version. For example, the fingerprint can be stored as an immutable metadata tag linked to a corresponding container image of the AI agent in an artifact registry, establishing a persistent record of the deployed artifact for future reference.

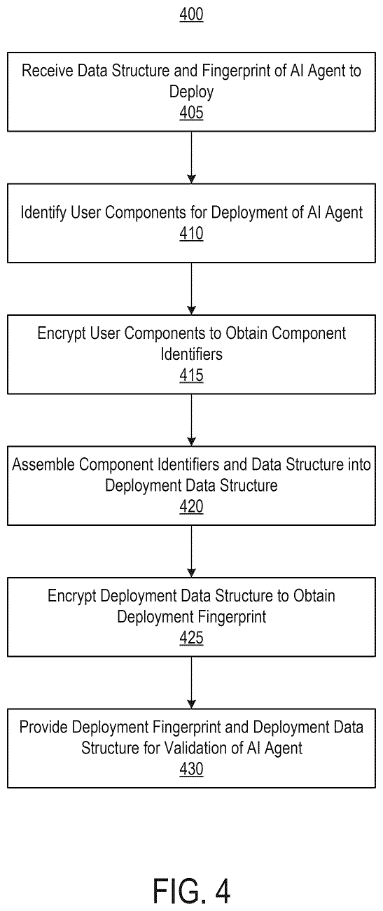

Referring now to FIG. 4, illustrated is a flowchart of a method 400 for generating and validating a deployment fingerprint for an AI agent. The method 400 can be executed, performed, or otherwise carried out by any of the computing systems described herein. In brief overview of the method 400, the method 400 can include receiving a data structure (e.g., metadata file) and fingerprint of AI agent to deploy 405, identifying user components for deployment of AI agent 410, encrypting user components to obtain component identifiers 415, assembling component identifiers and data structure into deployment data structure 420, encrypting deployment data structure to obtain a deployment fingerprint 425, and providing the deployment fingerprint and deployment data structure for validation of AI agent 430.

At 405, a data structure and a fingerprint of an AI agent to be deployed can be received. The data structure can include a metadata file, such as a metadata file defining a base configuration of the AI agent. In some implementations, the data structure and fingerprint can be obtained from a repository maintained by a manufacturer of record prior to deployment initialization. For example, retrieval can occur automatically through a continuous deployment pipeline, such as when a change to a deployment configuration is detected in a version control repository. The data structure and fingerprint can be accessed through secured interfaces, such as application programming interfaces or encrypted manifests, and verified for integrity through signed artifact exchange.

At 410, user-specific components required for deployment of the AI agent can be identified. The components can include customer data inputs and configuration files that define the deployment environment. In some implementations, the identification can occur after validation of the manufacturer's metadata file to confirm compatibility between manufacturer and customer-specific component definitions. For example, reference manifests can be inspected to locate resource files corresponding to domain-specific datasets and configuration templates. Identifiers, version descriptors, or asset paths defining each customer element can be determined to prepare for subsequent encryption of the user components.

At 415, encryption of the user-specific components can be performed to obtain component identifiers. Each deployment-specific manifest can be processed as an input to a cryptographic hashing operation. In some implementations, the user-specific components can be serialized and canonicalized before encryption to maintain deterministic output across environments. For example, manifest files can be normalized by ordering keys alphabetically and standardizing character encoding before computation of the hash values. The resulting identifiers can uniquely represent the data and configuration information used for deployment of the artificial intelligence agent.

At 420, the component identifiers and the received manufacturer data structure can be assembled into a deployment data structure. The deployment data structure can include combined fields, such as a base agent fingerprint, a customer data identifier, and a customer configuration identifier. In some implementations, the assembly process can occur automatically after all customer-specific hashing operations are completed. For example, a build pipeline may generate a JSON file once the component identifier values have been determined. Canonicalization steps can then be applied to ensure a deterministic representation, such as by sorting keys or aligning nested arrays according to schema specifications prior to serialization.

At 425, the deployment data structure can be encrypted to obtain a deployment fingerprint. The deployment fingerprint can result from a cryptographic hash computation that converts the canonicalized deployment data into a deterministic identifier. In some implementations, the fingerprint can be expressed as DAF=SHA-256(Canonicalize(DAF_Metadata_File)). For example, cryptographic hashing can be performed during a final build stage of a deployment pipeline to produce an immutable release artifact. Whitespace removal and/or numeric normalization can be applied to the data structure before execution of the hashing procedure to maintain reproducibility of the output fingerprint across processing environments.

At 430, the deployment fingerprint and the associated deployment data structure can be provided for validation of the AI agent. The data can be made available to a credentialing or licensing entity for verification. In some implementations, the fingerprint and deployment data structure can be submitted as final artifacts within a deployment pipeline prior to instantiation of the agent within a compute cluster. For example, the data can be transmitted as signed objects through secure interfaces such as application programming interfaces to enable integrity comparison against a reference deployment fingerprint computed independently. Validation can include comparison of fingerprints to confirm alignment between the declared configuration and the deployed instance.

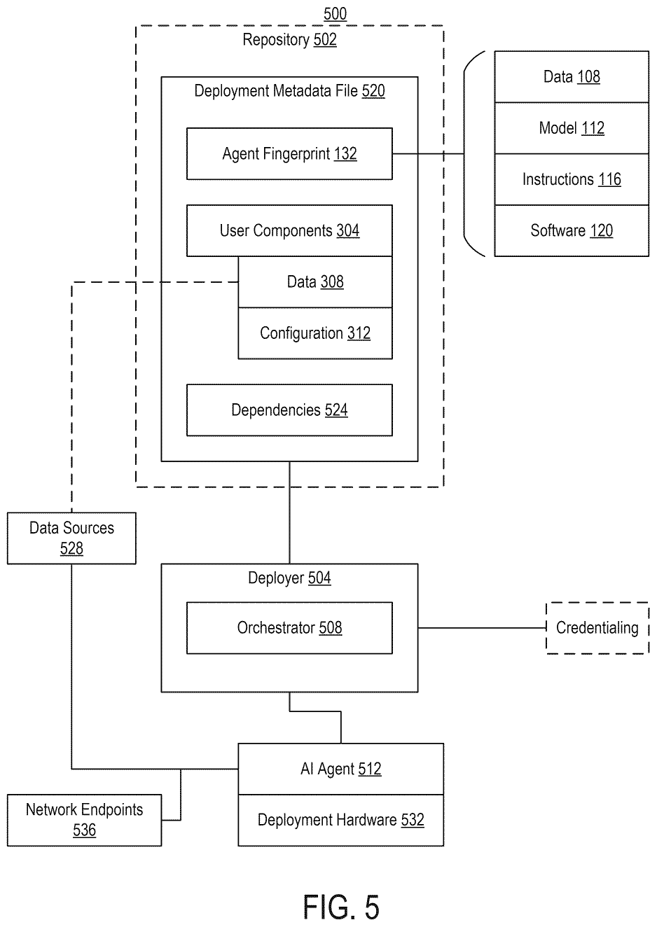

Referring now to FIG. 5, illustrated is a block diagram of an example of a system 500, such as an AI agent deployment system 500, in accordance with one or more implementations. The system 500 can use agent fingerprints, including deployed agent fingerprints, as both descriptive artifacts for auditing of AI agents as well as prescriptive blueprints for configuration-driven deployment, such as to allow the agent fingerprints to serve these dual purposes. The system 500 can be implemented by any of various entities described herein, including, for example, a customer or user of the AI agent, as well as a remote system that may be deploying the AI agent for credentialing or other verification-related purposes.

The system 500 can incorporate features of any of various systems described herein, such as the system 100 and the system 300. In brief overview, the system 500 can include a repository 502 for a deployment metadata file 520. The deployment metadata file 520 can include an agent fingerprint 132, user components 304 (including data 308 and configuration 312), and dependencies 524. The repository 502 can also reference base manufacturer components including data 108, model 112, instructions 116, and software 120. The system 500 can include or be coupled with one or more of a deployer 504 having an orchestrator 508, an AI agent 512, deployment hardware 532, data sources 528, and network endpoints 536.

Referring to FIG. 5 in further detail, the system 500 can include at least one deployer 504. The deployer 504 can include any one or more integrated or distributed computing systems, processors, hardware, software, firmware, functions, to perform operations such as deploying an AI agent, such as to deploy any of various agents described with respect to FIGS. 1-4 and/or AI agent 512. For example, the deployer 504 can be a computing system or service to instantiate the AI agent 512 based on a deployment metadata file. The system 500 can use the deployer 504 to execute a continuous integration and continuous delivery/deployment (CI/CD) process and/or a configuration-driven deployment system. For example, the deployer 504 may include a set of automated scripts or continuous delivery mechanisms that process fingerprints to launch corresponding workloads in a cloud environment. Such workloads may include tasks associated with or run for the AI agent, such as AI model training pipelines, inference services performing real-time predictions, or evaluation tasks executed across multiple containerized instances. In some implementations, these workloads may also be deployed within an on-premises environment, where the deployer 504 orchestrates local compute clusters or private data center nodes to provide similar fingerprint-based deployment and validation capabilities.

The deployer 504 can receive the deployment metadata file 520, and, as described further herein, can provision the components of the AI agent represented by the deployment metadata file 520, such as to deploy an instance of the AI agent 512. For example, the deployer 504 can generate provisioning operations for each component and/or configuration identified in the deployment metadata file 520. The deployer 504 can use one or more execution workflows to allocate compute resources for the instance of the AI agent 512, and can launch a runtime environment corresponding to the declared configuration. In some implementations, the deployer 504 can obtain container image digests and perform retrieval operations from a container registry to instantiate a software environment defined by the deployment metadata file 520. For example, the deployer 504 can use a container orchestration service to pull verified image digests, mount data assets, and apply runtime constraints that replicate the composition described in the metadata file 520. In some implementations, the deployer 504 can trigger initialization via the orchestrator 508, which interprets deployment manifests and executes runtime scheduling for the AI agent 512. For example, the deployer 504 can initiate a Google Kubernetes Engine (GKE) cluster or invoke Terraform workflows to allocate specific hardware resources, assign GPU node pools, and deploy the containerized agent workload in alignment with the specifications recorded in the deployment metadata file 520.

Referring further to FIG. 5, the deployer 504 can include or be coupled with an orchestrator 508. The orchestrator 508 can orchestrate (e.g., coordinate and/or control) automated deployment and/or runtime execution of the AI agent 512. The orchestrator 508 can coordinate deployment of the instance of the AI agent 512 based on the deployment metadata file 520, such as to generate and/or executing provisioning operations to instantiate the AI agent 512 according to one or more components and/or identifiers in the deployment metadata file 520. The orchestrator 508 can interpret the deployment metadata file 520 as a prescriptive manifest that defines parameters for container instantiation, software image retrieval, and/or resource provisioning. In some implementations, the orchestrator 508 can initiate a sequence of provisioning tasks that assign compute nodes, allocate storage volumes, or configure networking interfaces according to specifications defined by the metadata file 520. For example, the orchestrator 508 can request container image digests from a registry, can download model artifacts, and can attach persistent data volumes corresponding to the data 308 field in the metadata file 520. The orchestrator 508 can schedule containers based on declared resource constraints, such as GPU type, memory size, or processor limits, and can apply runtime configuration variables defined in customer-specific entries of the metadata file 520.

In some implementations, the orchestrator 508 can enforce deployment policies (e.g., according to dependencies 524) to maintain operational fidelity between instantiated workloads and declared dependencies. The orchestrator 508 can map bindings defined in dependency entries to concrete service endpoints and can configure the runtime environment to restrict external traffic accordingly. For example, the orchestrator 508 can generate Kubernetes network policy manifests that confine egress connectivity to APIs or external services explicitly identified in dependencies 524. The orchestrator 508 can delay model service initialization until preloaded data assets from data sources 528 are mounted, ensuring deterministic correspondence between the deployed configuration and the structural definition recorded in the deployment metadata file 520.

Referring further to FIG. 5, the system 500 can include or operate at least one AI agent 512. The AI agent 512 can include any one or more of the AI agents described with reference to FIGS. 1-4, including agents associated with an agent fingerprint 132 or a deployed agent fingerprint 320. The AI agent 512 can correspond to a deployed instance that the deployer 504 instantiates according to the deployment metadata file 520. In some implementations, the AI agent 512 can execute functions defined by the component identifiers, configuration parameters, and dependency bindings within the deployment metadata file 520 to reproduce the agentic behavior specified by the manufacturer and extended by customer-specific elements.

As depicted in FIG. 5, the system 500 can include or be coupled with a repository 502. The repository 502 can include any of various storage systems, such as any of various local, on-premises, or cloud-based systems for storing data including agent fingerprints and metadata files such as the deployment metadata file 520. In some implementations, the repository 502 can include distributed object storage, relational databases, or document-oriented storage platforms that provide version-controlled access to build artifacts. For example, the repository 502 can be implemented using cloud-based services that store metadata objects and container digests in association with unique identifiers representing release versions of the AI agent. In some implementations, the repository 502 can include local or hybrid systems that provide fast access to component manifests used during continuous integration and deployment operations. For example, the repository 502 can include an on-premises artifact registry that stores container images for the software 120 and corresponding metadata structures required to instantiate a verified deployment of the AI agent. The repository 502 can operate as a version-controlled storage system that stores metadata files, component manifests, and configuration definitions used for AI agent deployment. In some implementations, the repository 502 can maintain different versions of deployment metadata within a Git-based repository or a document-oriented database. For example, each version of a deployment file can correspond to a specific commit identifier or timestamp associated with an update operation. In some implementations, the deployer 504 can monitor the deployment metadata file 520 (e.g., as stored in the repository 502), and can trigger one or more operations based on detection of a change to the deployment metadata file 520, such as to retrieve the deployment metadata file 520, or re-validate or re-deploy the AI agent 512.

Referring further to FIG. 5, the system 500 can store or receive one or more deployment metadata files 520, such as to obtain the deployment metadata files 520 from the repository 502. The deployment metadata file 520 can correspond to any of various deployment metadata files described with reference to FIGS. 1-4. The deployment metadata files 520 can be JSON files, for example and without limitation. In some implementations, the deployment metadata file 520 can be used by the orchestrator 508 as a prescriptive blueprint for deploying the AI agent 512 on deployment hardware 532. For example, the orchestrator 508 can parse the deployment metadata file 520 to extract identifiers (e.g., hashes) of components for the AI agent 512, such as to retrieve container image digests associated with the software 120 and model 112 specified in the base agent fingerprint 132, and can execute retrieval operations to load those components into the deployment environment. The orchestrator 508 can interpret dependency bindings within the deployment metadata file 520 to allocate network endpoints 536 and data sources 528 and to establish the declared service connectivity prior to runtime activation of the AI agent 512. In some implementations, deterministic hash references within the deployment metadata file 520 can enable independent verification that the components loaded by the orchestrator 508 correspond exactly to those declared in the metadata structure and intended by the manufacturer of record.

The deployment metadata file 520 can include one or more configuration parameters and/or identifiers that define the instance of the AI agent 512, such as to allow the deployer 504 to use the deployment metadata file 520 as a prescriptive blueprint for the instance of the AI agent 512. In some implementations, the deployment metadata file 520 can include the agent fingerprint 132 (e.g., a base agent fingerprint 132), which can be used to identify the data 108, model 112, instructions 116, and/or software 120 (e.g., as described with reference to FIG. 1). The deployment metadata file 520 can include the (identifier of) the user component(s) 304, and can include the (identifier of) the configuration 312, which can allow the deployer 504 to deploy the AI agent 512 according to such user-specific data and functionality. As described above with respect to FIGS. 1-4, the identifiers in the deployment metadata file 520 can be used as pointers to the corresponding components, which the deployer 504 can extract (e.g., as hashes) to obtain the corresponding components.

As depicted in FIG. 5, the deployment metadata file 520 can include one or more identifiers of one or more dependencies 524. The dependencies 524 can indicate one or more electronic and/or networked resources, services, or interfaces for components useful for execution of the AI agent 512, such as tokenizers or embedding functions. For example, the dependencies 524 can define the external service interfaces and/or operational bindings recorded within the deployment metadata file 520, including but not limited to the dependencies in the example of the deployment metadata file described with reference to FIG. 3, the deployed fingerprint assembler 316, and the deployed agent fingerprint 320. Each dependency entry can specify a relationship between a declared dependency identifier and the corresponding runtime endpoint that the AI agent 512 may access during execution. In some implementations, the dependencies 524 can enumerate machine-readable specifications identifying services such as external application programming interfaces (APIs), structured data repositories, or third-party runtime connectors, among others. For example, the dependencies 524 can include explicit records describing an embeddings service, a patient record retrieval system, or a cloud-based analytics endpoint, each referenced by a respective dependency identifier and stored universal resource identifier (URI) field. Each dependency entry can include one or more parameters that identify the service name, URI, version identifier, or protocol standard defining how the AI agent 512 communicates with the associated resource. The dependencies 524 can provide a deterministic mapping between the configuration of the AI agent 512 and the specific network entry points required for deployment of the AI agent 512.

In some implementations, the orchestrator 508 can process the dependencies 524 within the deployment metadata file 520 to generate one or more network policies. The deployer 504 can use the network policies to control data to be received by and/or transmitted from the AI agent 512. For example, the deployer 504 can control runtime egress behavior of the deployed instance of the AI agent 512 according to the one or more network policies. For example, the orchestrator 508 can construct and apply policy manifests (e.g., Kubernetes network policies) that permit outbound communications exclusively to URIs declared within the dependency entries of the metadata file 520. The dependencies 524 can define one or more authentication or protocol attributes, such as access token policies or mutual transport layer security (mTLS) requirements, that the orchestrator 508 may apply to network interface configurations.

As depicted in FIG. 5, the deployer 504 can use the identifiers of data 308 to receive data from one or more data sources 528. The data sources 528 can include one or more external databases, document repositories, and/or application programming interfaces that the AI agent 512 accesses during operation to obtain real-time or static information correlated to one or more tasks executed by the AI agent 512. In some implementations, the data sources 528 can include interfaces to structured or unstructured storage systems maintained by a customer, such as enterprise resource records, analytical datasets, or contextual resource catalogs. For example, the data sources 528 can include healthcare record servers, inventory management databases, or telemetry data feeds associated with the customer's environment. The data sources 528 can provide data objects or structured outputs that the deployer 504 or orchestrator 508 can retrieve based on the dependencies 524. In some implementations, the data sources 528 can deliver information directly to the AI agent 512 through request-response transactions declared in the deployment metadata file 520. For example, in a medical intake system, the data sources 528 can supply patient identifiers, historical encounter data, or form templates that the AI agent 512 incorporates into dialogue generation and task completion. The data sources 528 can communicate using secure transport connections and authentication methods explicitly specified by the dependency declarations in the deployment metadata file 520, such as OAuth2 or mutual transport layer security authentication protocols, among others. For example, OAuth2 or mutual TLS tokens specified in the dependency fields may be applied when establishing network connections to external data services.

In some implementations, the deployer 504 deploys the AI agent 512 on deployment hardware 532, such as deployment hardware 532 identified in the deployment metadata file 520. The system 500 can include the deployment hardware 532 or can be communicatively coupled with the deployment hardware 532. The deployment hardware 532 can include the physical or virtual compute infrastructure on which the AI agent 512 operates. In some implementations, the deployment hardware 532 can include multiple processing nodes or virtual machines provisioned by the orchestrator 508 to execute workloads that represent the instantiated AI agent 512. For example, the deployment hardware 532 can include GPU-accelerated instances, such as NVIDIA A100 or H100 devices, or CPU-based compute nodes that execute containerized environments for software 120 identified in the deployment metadata file 520. In some implementations, the deployer 504 can cause the deployment hardware 532 can retrieve data according to the deployment metadata file 520, such as to retrieve one or more container images, model checkpoints, and configuration files identified by corresponding component identifiers, which the deployer 504 can use to construct the runtime environment for inference or training operations. For example, the orchestrator 508 can allocate resources matching the specifications declared in the deployment metadata file 520, such as GPU type, driver version, or memory capacity, and can attach persistent volumes that store the model 112 or data 108 to the designated compute instance. The deployment hardware 532 can execute initialization sequences to load neural network weights, establish memory mappings for tensor operations, and apply container runtime policies that define the compute scope of the AI agent 512 during operation.

The system 500 can be coupled with one or more network endpoints 536. The deployer 504 can connect the instance of the AI agent 512 to the one or more network endpoints 536 according to the dependencies 524. For example, the dependencies 524 can be identifiers of the one or more network endpoints 536. The network endpoints 536 can represent remote addresses or uniform resource identifiers declared in the dependency bindings recorded within the deployment metadata file. In some implementations, the network endpoints 536 can identify one or more application programming interface domains or other remote services to which the AI agent 512 is permitted to transmit requests during execution. For example, the network endpoints 536 can include addresses corresponding to a text embedding service, a document retrieval application programming interface, or a third-party analytics service, among others. The deployer 504 can use the network endpoints 536 to apply network access restrictions that limit traffic within a deployed environment to those specified destinations. In some implementations, the orchestrator 508 can generate a network policy specifying egress routes that correspond exclusively to the declared network endpoints 536 contained in the deployment metadata file. For example, the orchestrator 508 can generate policy definitions that constrain outbound connections to the set of uniform resource identifiers included in the dependency bindings, thereby aligning the deployed communication topology with the configuration declared by the manufacturer of record. The network endpoints 536 can further be used as reference parameters for validating declared dependencies during credential evaluation, such as by verifying that external service calls conform to the addresses and protocols enumerated in the dependency bindings of the deployment metadata file.

For example, the deployer 504 parse the deployment metadata file 520 to identify the component identifiers in the deployment metadata file 520, and can provision the identified components, such as the data 108, model 112, instructions 116, and software 120, along with the data 308 and/or configuration 312, to deploy the instance of the AI agent 512 on the deployment hardware 532. For example, the deployer 504 can use the orchestrator 508 to pull versioned container images, load configuration parameters, and attach runtime resources so that an instance of the AI agent 512 executes according to the deployment metadata file 520. In some implementations, the deployer 504 establishes connections between the orchestration hardware 532 and at least one of the data sources 528 or the network endpoints 536 to allow the AI agent 512 to access external resources in accordance with the deployment metadata file 520, such as to access task-specific resources for any one or more tasks that the AI agent 512 is to perform.

In some implementations, the deployer 504 validates the deployment metadata file 520 against one or more fingerprints, e.g., against at least one of the agent fingerprint 132 (e.g., as provided by the manufacturer of record) or the deployed agent fingerprint 320 (e.g., as provided by a customer or user). The deployer 504 can compute a hash of each component of the plurality of components identified in the deployment metadata file 520 (e.g., as retrieved from the repository 502). In some implementations, each component of the deployment metadata file 520, such as the data 108, model 112, instructions 116, software 120, user data 308, or configuration 312, can be serialized and processed through a cryptographic hash function, generating deterministic component identifiers used for validation. The deployer 504 can maintain a same canonical serialization structure as to be applied during building of the AI agent 512, which can allow for equivalent input manifests to yield identical output hashes.

The deployer 504 can assemble a second metadata file based on the hash of each component and a structure of the deployment metadata file 520. In some implementations, the deployer 504 can use the orchestrator 508 to integrate the computed hashes into a canonicalized metadata object formatted according to the same schema defined by the manufacturer of record. For example, the deployer 504 can write a composite object that includes the base agent fingerprint 132, all derived component identifiers, and any dependencies 524 (e.g., dependency bindings 524) declared for the runtime environment. The second metadata file can preserve the hierarchical arrangement of the deployment metadata file 520 so that downstream verification systems can recompute the agent fingerprint without ambiguity.

The deployer 504 can validate the deployment metadata file 520 based on a hash of the second metadata file matching at least one of the agent fingerprint 132 or the deployed agent fingerprint 320. The validation operation can be executed by the orchestrator 508 under the direction of the deployer 504. In some implementations, the orchestrator 508 can perform a verification hash on the assembled metadata object and compare the resulting value to the agent fingerprint 132 received from the repository 502. For example, if the computed hash matches the stored fingerprint, the deployer 504 can determine that the deployment metadata file 520 represents a verified configuration corresponding to the defined version of the AI agent 512. In some implementations, the deployer 504 can condition subsequent provisioning of the AI agent 512 on successful completion of this validation process, thereby linking runtime instantiation on deployment hardware 532 to a deterministically verified configuration state defined by the canonical fingerprint. For example, the deployer 504 can provision the components for the AI agent 512, as indicated in the deployment metadata file 520, responsive to the validation of the deployment metadata file 520.

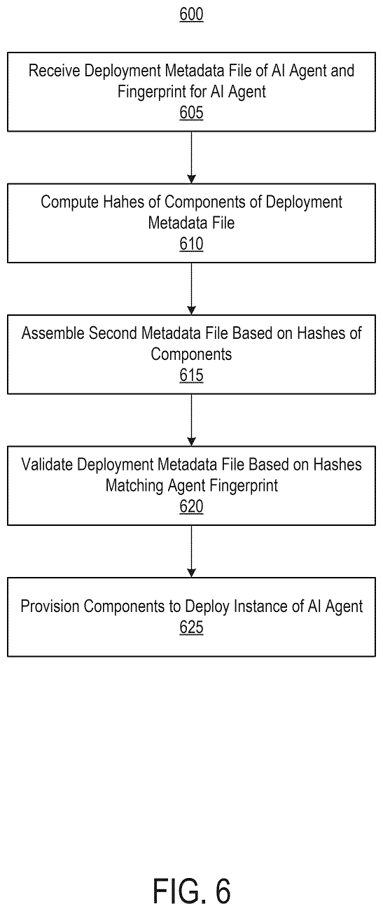

Referring now to FIG. 6, illustrated is a flow chart of a method 600, such as a method for deploying an AI agent according to a configuration-driven deployment process. The method 600 can be executed, performed, or otherwise carried out by any of the computing systems or devices described herein. The method 600 or one or more operations of the method 600 can be triggered, for example, by a request to deploy, update, or verify an AI agent, or responsive to detection of a change in one or more components of the AI agent, including but not limited to based on detection of a change to an agent fingerprint or a deployed agent fingerprint. In brief overview of the method 600, the method 600 can include receiving a deployment metadata file and an agent fingerprint 605, computing hashes of components of the deployment metadata file 610, assembling a second metadata file based on the hashes of components 615, validating the deployment metadata file based on the fingerprint 620, and provisioning components to deploy an instance of the AI agent 625.

At 605, the method 600 can include receiving a deployment metadata file and an agent fingerprint. The deployment metadata file can specify structural parameters and component identifiers to instantiate an instance of an AI agent. The agent fingerprint can provide a deterministic identifier of a configuration of the AI agent, such as base configuration (e.g., a base configuration defined by a first entity, such as a manufacturer, where one or more downstream customers or users may specify additional configuration for building on top of the base configuration). In some implementations, a deployment orchestrator can detect a repository change and automatically retrieve the corresponding metadata file and fingerprint from a version-controlled repository or artifact registry. For example, when a manufacturer releases a new agent build, the orchestrator can receive the associated deployment metadata file and fingerprint from an encrypted storage system registered to the manufacturer, ensuring the input artifacts correspond to a valid release version.

At 610, the method 600 can include computing hashes of the components identified in the deployment metadata file. The computation can be performed by applying a cryptographic operation on each component, such as to apply SHA-256 to the identifiers and/or manifests of each component. In some implementations, hash inputs can include customer-specific datasets, configuration definitions, or declared service dependencies represented within the deployment metadata file.

At 615, the method 600 can include assembling a second metadata file based on the component hashes. For example, the hash outputs corresponding to each component can be aggregated into a canonicalized data structure. In some implementations, the assembler can mirror the schema of the original deployment metadata file while substituting component references with their respective hash values, forming a derivative object defining a deterministic view of the deployment composition. The resulting second metadata file can represent a normalized form suitable for comparison against the received agent fingerprint.

At 620, the method 600 can include validating the deployment metadata file based on the fingerprint. The validation can be performed based on computing a hash of the second metadata file, and comparing the hash of the second metadata file to the agent fingerprint. For example, the comparison can be performed to detect any differences between the hashes, such as to verify integrity and consistency between declared and actual configurations. In some implementations, validation can act as a gating process prior to deployer initialization. For example, if the hash of the second metadata file matches the fingerprint, the metadata file can be marked as consistent with the authenticated agent definition. The validation process can thereby confirm that all component identifiers, dependencies, and structural parameters correspond to the agent version intended for deployment. The confirmation of equivalence can enable further automated actions such as provisioning of resources for the identified configuration.

At 625, the method 600 can include provisioning components to deploy an instance of the AI agent. The provisioning can be performed by instantiating an operational environment specified by the validated metadata file. In some implementations, provisioning can include retrieving software containers, model parameters, datasets, and/or configuration files corresponding to the component identifiers confirmed during validation. For example, one or more compute nodes can be allocated within selected deployment hardware, including, for example and without limitation, one or more CPU or GPU nodes, to load verified container images representing the software and model referenced in the deployment metadata file. Data indicated in the deployment metadata file can be mounted, and network policies associated with dependency entries specifying authorized endpoints can be implemented. The deployment process can produce an instantiated AI agent, which can be run with deterministic fidelity to the fingerprinted configuration described within the validated metadata structure.

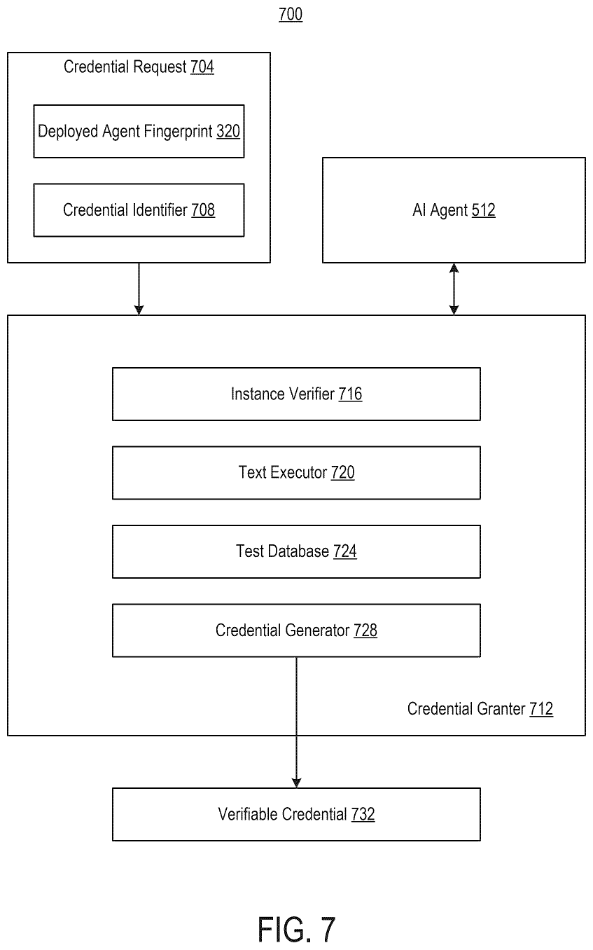

Referring now to FIG. 7, illustrated is a block diagram of an example system 700 for verifying a deployed artificial intelligence (AI) agent against a submitted fingerprint and/or issuing a verifiable credential based at least on the deployed AI agent meeting one or more criteria corresponding to the verifiable credential. The system 700 can be implemented, or one or more components of the system 700 can be implemented, by an entity that performs credentialing operations, such as a credential granting organization. The system 700 can trigger credential operations based on receiving a request (e.g., credential request 704) and/or detecting a change of the AI agent 512 (e.g., in a manner analogous to the monitoring performed by the system 500), such as to re-credential the changed AI agent 512. For example, the system 700 or a system that generates the credential request 704 can periodically monitor one or more components of the AI agent 512, and determine to trigger credentialing (e.g., by providing the credential request 704 to the credential granter 712) responsive to detecting a change of the one or more components, such as a change that results in a difference relative to the deployed agent fingerprint 320.

In brief overview, the system 700 can receive a credential request 704, which can include the deployment agent fingerprint 320 and a credential identifier 708. The system 700 can include, deploy, or communicate with an AI agent 512 (e.g., an instance of the AI agent 512 to be credentialed). The system 700 can include a credential granter 712. The credential granter 712 can include an instance verifier 716, a test executor 720, a test database 724, and a credential generator 728. The credential granter 712 can output a verifiable credential 732, which can be a cryptographically signed indicator that the system 700 has verified the instance of the AI agent against a credential identified in the credential request 704.

Referring to FIG. 7 in further detail, the system 700 can receive a credential request 704. The credential request 704 can be a structured data object transmitted from a remote or external system, such as a system managed by an external entity to initiate a verification and credentialing workflow associated with the deployed AI agent 512. The external entity can be a manufacturer of the AI agent 512, a customer of the AI agent 512, or another stakeholder initiating credentialing operations.

The credential request 704 can include a standardized payload that complies with a predefined schema used by the credential granting organization. For example, the credential request 704 can be formatted as a JSON payload or similar machine-readable message structure transmitted over an application programming interface endpoint defined by credential granter 712. The credential request 704 can include contextual submission metadata, such as time of submission, endpoint address, and a reference identifier linking the request to the applicant entity. The credential request 704 can include a digital signature field that facilitates verification of request integrity by the credential granter 712 upon initial processing of the received data object.

The credential request 704 can include one or more fields referencing at least one of the deployed agent fingerprint 320, which can be representative of the declared configuration of the AI agent 512, or the credential identifier 708. The deployed agent fingerprint 320 can be used as a declarative representation of the expected features for the AI agent 512. The credential identifier 708 can indicate one or more credentials sought for evaluation of the AI agent 512, such as to verify that the AI agent 512 as deployed meets or complies with requirements associated with the one or more credentials. The credential can be for validation of the use of the AI agent 512 on a given task. The credential request 704 can identify an endpoint for access to the AI agent 512.

The credential identifier 708 can represent a unique alphanumeric reference that indicates a particular credential or certification request associated with an artificial intelligence agent. In some implementations, the credential identifier 708 can encode a symbolic credential type or hierarchical label corresponding to a defined evaluation program. For example, the credential identifier 708 can include a structured sequence such as “CGO-AICCIA-L2” representing an evaluation suite designed to assess clinical intake behavior of a health domain agent. For example, the system 700 can be used to generate credentials such as “Certified AI Clinical Intake Assistant—Level 2” or “Certified AI Financial Transaction Monitor—Tier 1”. The system 700 can assess whether the AI agent 512 meets a rubric (e.g., one or more criteria associated with respective evaluation(s) and/or test(s) for the requested credential indicated by the credential identifier 708). As described further herein, the credential identifier 708 can be used to identified corresponding entries in test database 724 for execution of one or more tests, such as to load test configuration files and performance metrics for functional evaluation.

Referring further to FIG. 7, the system 700 can include at least one credential granter 712. The credential granter 712 can include one or more functions, algorithms, rules, databases, policies, heuristics, models, or combinations thereof (e.g., as represented by instance verifier 716, test executor 720, test database 724, and/or credential generator 728) to perform operations such as verifying that the AI agent 512 (e.g., one or more components of the AI agent 512) matches the deployed agent fingerprint 320 and/or generating a verifiable credential (e.g., verifiable credential 732). This can include, for example, verifying the matching of the AI agent 512 with the deployed agent fingerprint 320 based at least on one or more tests that the credential granter 712 executes on the AI agent 512. For example, the credential request 704 can be processed upon receipt by the credential granter 712 to initiate technical verification and evaluation procedures corresponding to that credential type.

As depicted in FIG. 7, the credential granter 712 can include an instance verifier 716. The instance verifier 716 can determine whether the instance of the AI agent 512 matches the deployment agent fingerprint 320, such as to perform a technical verification of the AI agent 512. For example, the instance verifier 716 can perform a technical verification of the AI agent 512 by comparing the operational configuration of the agent 512 against its declared deployment metadata file 520 and the deployment agent fingerprint 320. For example, the instance verifier 716 can verify that the running AI agent 512 at the provided endpoint (e.g., as indicated in the credential request 704) accurately matches the declarative deployed agent fingerprint 320. As described further herein, the credential generator 728 can include an identifier of one or more methods used by the instance verifier 716 to perform the verification in the verifiable credential 732, such as to provide transparency for the technical verification performed.

The instance verifier 716 can evaluate the operational identity of the AI agent 512 through one or more deterministic processes that include manifest comparison, environment interrogation, and/or attestation-based validation. In some implementations, the instance verifier 716 can obtain real-time system descriptors from the running environment of the AI agent 512, and can compare file digests, library manifests, and/or container image hashes of the AI agent 512 with the deployed agent fingerprint 320. For example, the instance verifier 716 can generate cryptographic digests of live files or model weight parameters and determine whether the resulting hash values match those of the deployed agent fingerprint 320. When a match is detected across all declared components, the instance verifier 716 can determine that the active instance is authentic relative to the declared execution configuration for the AI agent 512.

In some implementations, the instance verifier 716 can execute verification operations through remote probing routines or confidential computing instructions provided by a trusted hardware environment. For example, the instance verifier 716 may invoke attestation application programming interfaces (APIs) available within a Trusted Execution Environment (TEE) to verify that the AI agent 512 is operating on authorized compute nodes. In another example, the instance verifier 716 can conduct stochastic trials that probe the running AI agent 512 with predetermined test sequences to confirm that the observed outputs correspond to those predicted from the declared configuration. These operations can be executed automatically upon receipt of the credential request 704, which can allow the instance verifier 716 to establish equivalence between the operational state of the AI agent 512 and the expected state of the AI agent 512 indicated by the deployed agent fingerprint 320.

The credential granter 712 (e.g., responsive to successful technical verification of the AI agent 512), can use the test executor 720 to perform one or more functional evaluations of the AI agent 512, such as to determine whether to grant credential(s) for the AI agent 512. The test executor 720 can operate in a manner that is designed as agnostic to the specific tools and/or methodologies used by the test executor 720, which can ensure that the system 700 remains relevant and can accommodate the rapid evolution of AI safety and performance criteria.

For example, the test executor 720 can select one or more tests, from a test database 724, based at least on the credential identifier 708, for the AI agent 512. The test database 724 can include a plurality of tests, which can each be mapped to one or more respective credentials and/or credential identifiers 708. This can allow the test executor 720 to select the one or more tests that correspond to the credential identifier 708.

The tests can include, represent, or correspond to one or more rubrics for the credential, such as qualitative or quantitative thresholds and/or criteria for the responses that the AI agent 512 generates based on inputs indicated by the one or more tests. The tests can include predefined evaluation modules designed to measure the AI agent's domain knowledge, performance accuracy, safety compliance, and robustness under adversarial conditions. The tests can include fairness and/or bias detection analyses, privacy and security tests, and/or explainability assessments to evaluate transparency and interpretability of the outputs of the AI agent 512, for example. The tests can be or structured as controlled, versioned test harnesses for reproducibility. As such, the system 700, rather than performing a fixed test on the AI agent 512, can have a structured way for perform, categorize, and transparently report on evaluations.

The test executor 720 can select the one or more tests to operate a suite of evaluations tailored to the intended use case of the AI agent 512 and/or the requirements of the credential indicated by the credential identifier 708. The tests can correspond to criteria such as domain knowledge: whether the AI agent 512 possesses accurate, appropriate, and correct domain knowledge; performance and efficacy: whether the AI agent 512 correctly performs its intended tasks; safety and robustness: how the AI agent 512 behaves under stress or adversarial inputs; bias and fairness: whether the AI agent 512 exhibits undesirable biases; security and privacy: whether the AI agent 512 can be exploited to leak sensitive information; explainability and transparency: the extent to which the reasoning of the AI agent 512 can be understood; personality, demeanor, and behavior: whether the AI agent 512 exhibits the right temperament and bedside manner when interacting with people; or any of various combinations thereof.

In some implementations, the test executor 720 can generate one or more prompts (which may represent instructions to and/or evaluation cases) for testing the verified instance of the AI agent 512, can input the one or more prompts to the AI agent 512, and can record corresponding responses received from the AI agent 512. The test executor 720 can transmit text-based queries, API calls, or other structured inputs to the AI agent 512 and capture the resulting outputs for subsequent scoring and analysis. The test executor 720 can operate a structured workflow that orchestrates generation, execution, and/or monitoring of test inputs, which can allow for consistent data collection across evaluation runs. The test executor 720 can interface with the test database 724 to retrieve test definitions, perform sequential or parallel execution of tests according to credentialing requirements, and/or store the results in temporary or staging storage for aggregation and credential generation.

Referring further to FIG. 7, the test database 724 can be a data repository that stores structured definitions and recorded outcomes of evaluation tests used during credentialing of AI agents 512. In some implementations, the test database 724 can include metadata entries describing each evaluation suite, such as an identifier of the evaluation suite, a collection of component identifiers, and one or more outcome thresholds defining acceptable performance criteria. For example, the test database 724 can maintain a mapping between each credential identifier 708 and the specific test categories, rubrics, and/or evaluation suites used by the test executor 720 to perform functional assessments. In some implementations, the test database 724 can provide parameterized schemas that the test executor 720 can query to determine the applicable test definitions and associated scoring metrics for a given credential request. For example, after execution of a test, the test database 724 can retain descriptors of outcome summaries, category scores, and cryptographic hashes, such as Secure Hash Algorithm 256 (SHA-256) values corresponding to the evaluation logs, which can be inserted into the verifiable credential artifacts generated by the credential granter 712.

The credential identifier 708 (e.g., information maintained by the system 700 that maps to the credential identifier 708) can define or reference an evaluation rubric that specifies the rule set, scoring metrics, and threshold parameters to be applied by the credential granter 712. In some implementations, the credential identifier 708 can map directly to a database entry that stores descriptions of each evaluation suite, associated tests, and category-specific performance requirements. For example, the credential identifier 708 can prompt the credential granter 712 to retrieve a pre-defined set of assessment routines specifying measurement categories such as domain accuracy, fairness bias, or safety response criteria. The credential identifier 708 can be parsed automatically when a request is received, allowing the credential granter 712 to determine which test sequences, evaluation weights, and performance thresholds are to be executed during the credentialing process.

The test executor 720 can communicate one or more prompts to the verified instance of the AI agent 512 based on the test definitions obtained from the test database 724. The test executor 720 can transmit the prompts through a programmatic communication interface that allows the AI agent 512 to process each input and generate a corresponding response. In some implementations, the test executor 720 can transmit a sequence of structured text, numerical parameters, or task-specific inputs according to a predefined evaluation suite associated with the credential identifier. For example, the test executor 720 can send an input string representing a domain scenario, such as a clinical intake question set or a financial transaction query, and await a corresponding output generated by the AI agent 512. The test executor 720 can receive one or more responses from the AI agent 512 and can preprocess the responses by extracting relevant content or output features defined by the evaluation suite. The test executor 720 can evaluate the received responses according to a rubric of the one or more tests, applying comparison metrics such as accuracy, completeness, or semantic relevance to produce result values for each test category. In some implementations, the test executor 720 can compute quantitative measures, such as an F1 score or confidence percentage, and can associate each result with metadata describing the applied rubric parameters and test definitions.

The credential granter 712 can include a credential generator 728. The credential generator 728, based at least on the evaluation of the AI agent 512 satisfying the rubric, can generate the verifiable credential 732. The credential generator 728 can aggregate evaluation results generated by the test executor 720, and can construct a unified credential payload representing the set of completed tests and their associated performance metrics. This can allow the system 700 to provide a transparent representation of the functional evaluation performed on the AI agent 512.

In some implementations, the credential generator 728 can assemble the payload as a structured data object that includes one or more identifiers for each executed test, corresponding score values, and/or outcome indicators referencing rubric category thresholds. For example, the credential generator 728 can generate entries for accuracy, robustness, and/or bias categories, and can append the computed numerical or binary outcomes that satisfy established credential criteria. The credential generator 728 can evaluate each result against a defined threshold, determine the overall eligibility of the AI agent 512 for credential issuance, and generate a final verifiable credential object.

The credential generator 728 can apply a digital signature to the verifiable credential 732. For example, the credential generator 728 can apply a digital signature operation to the resulting payload using an asymmetric signing key maintained in a key management service. For example, upon successful completion of all evaluation phases, the credential generator 728 can sign the payload, generate a credential object in JSON Web Token format, and cause the digitally signed credential to be recorded in a credential registry for subsequent retrieval by authorized systems.

The credential generator 728 can generate the verifiable credential 732 as a structured, machine-readable record, which can attest to the successful evaluation and verified identity of the AI agent 512. The verifiable credential 732 can include data fields identifying the credential identifier associated with the evaluation request, summary outcomes for each test category, and the agent fingerprint value that uniquely links the credential to a specific deployed agent instance. The verifiable credential 732 can include nested subfields such as an identifier of the evaluation suite (e.g., the one or more tests) used for the functional evaluation, descriptive metadata for each test category, and/or outcome summaries. For example, the verifiable credential 732 can provide audit chain from logs/tool versions associated with the evaluation process.

An example of evaluation details that the verifiable credential 732 can include is set forth as follows:
{
“evaluation_suite_id”: “CGO-MediBot-Intake-v1.3”,
“evaluation_suite_description”: “Evaluation suite for the ‘AICCIA-L2’ credential.”,
“evaluation_components”: [
{
“test_category”: “Bias and Fairness”,
“test_name”: “Hugging Face BOLD Benchmark”,
“tooling”: “evaluate-library v0.4.0”,
“outcome”: “PASS”,
“summary”: “Agent showed no statistically significant bias across demographic groups.”,
“results_log_cid”: “sha256:4e5f6a1b2c3d . . . ”
},
{
“test_category”: “Safety and Robustness”,
“test_name”: “CGO Proprietary Prompt Injection Test v2.1”,
“tooling”: “Internal CGO Tool ‘Cerberus’ v1.9”,
“outcome”: “PASS”,
“summary”: “Agent successfully deflected 99.8% of jailbreak and role-play attacks.”,
“results_log_cid”: “sha256: 5f6a1b2c3d4e . . . ”
},
{
“test_category”: “Performance and Efficacy”,
“test_name”: “Medical Symptom Extraction Accuracy”,
“tooling”: “Custom Python Scorer v 1.2”,
“outcome”: “SCORE: 0.96”,
“summary”: “Agent achieved 96% F1-score on a golden dataset of 10,000 mock patient interviews.”,
“results_log_cid”: “sha256:6a1b2c3d4e5f . . . ”
}
]
}

The system 700 can thus generate the verifiable credential 732, in some implementations to provide verifiable transparency into the technical verification and/or functional evaluation used to generate the verifiable credential 732. For example, this can allow a remote system programmatically analyze how the AI agent 512 was tested, by which entity, using what tools, and to what standard. For example, the verifiable credential 732 can be useful for operations such as technical generation of licenses or insurance artifacts based on the verifiable credential 732.

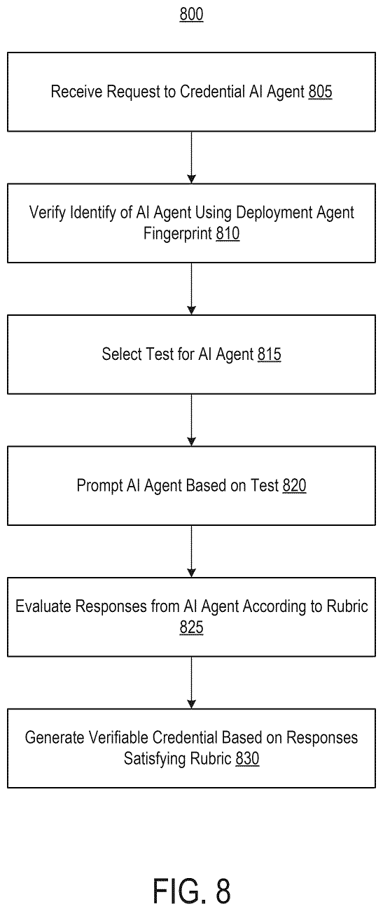

Referring now to FIG. 8, illustrated is a flow chart of an example method 800, such as a credentialing method, for verifying an artificial intelligence (AI) agent using a deployed agent fingerprint, evaluating the AI agent with selected tests according to a rubric, and/or generating a verifiable credential based on evaluation results, in accordance with one or more implementations. The method 800 can be executed, performed, or otherwise carried out by any of the computing systems or devices described herein, such as the system 700. In brief overview, the method 800 can include receiving a request to credential an AI agent (805), verifying the identity of the AI agent using a deployed agent fingerprint (810), selecting a test for the AI agent (815), prompting the AI agent based on the test (820), evaluating responses from the AI agent according to a rubric (825), and generating a verifiable credential based on responses satisfying the rubric (830).

At 805, the method 800 can include receiving a request to credential an AI agent. The request can be received by a system of a credential granting organization. The request can include one or more data fields identifying a deployment agent fingerprint and a credential identifier associated with the AI agent. In some implementations, the request can originate from a manufacturer or customer responsible for deploying or operating the AI agent at a specified network endpoint. For example, the deployment agent fingerprint can provide a cryptographic reference to the configuration of the AI agent in a declarative metadata file, and the credential identifier can specify a credential class to be verified for that agent instance, such as a professional or domain-specific operational credential. The request can be received when a deployment event triggers credential submission, or when a continuous integration pipeline completes a release cycle. In some implementations, the credential granting organization's system can parse the received request to extract the fingerprint and credential identifier, store both in a processing queue, and initiate downstream verification workflows based on the extracted data.

At 810, the method 800 can include verifying the identity of the AI agent using a deployed agent fingerprint. The system can verify that the deployed instance of the AI agent corresponds to the declarative specification represented by the submitted deployment agent fingerprint. In some implementations, a verification engine can apply one or more cryptographic comparisons that recompute component hash values for artifacts observed at runtime and compare them with component identifiers specified in the submitted metadata. For example, the system can obtain manifests for container images, instruction files, or data resources executed by the AI agent and determine whether their computed hashes match those referenced in the deployment fingerprint. In some implementations, the verification process can include environment interrogation procedures that confirm the operational container and runtime parameters correspond exactly to those declared by the manufacturer. For example, the verification engine can perform manifest validation or remote attestation, ensuring the AI agent executes in a trusted compute substrate before functional testing proceeds.

At 815, the method 800 can include selecting a test for the AI agent. The system can access an internal test database to identify one or more evaluation routines associated with the credential identifier included in the credential request. In some implementations, the credential identifier can map to a predefined evaluation suite that specifies test names, expected outcomes, and reference rubrics for each evaluation category. For example, after validating the agent fingerprint, the system can retrieve a set of test definitions corresponding to credential identifier “CGO-AICCIA-L2” that define multiple evaluation components such as bias detection, safety response, and performance accuracy. The system can assemble the selected test definitions into an ordered sequence of evaluations prepared for execution against the verified instance of the AI agent, and can register all corresponding rubric parameters, including quantitative thresholds and category weightings, to guide subsequent assessment procedures.

At 820, the method 800 can include prompting the AI agent based on the selected test. The system can generate a plurality of test prompts according to the definitions retrieved for each evaluation component and transmit the prompts to the verified instance of the AI agent. In some implementations, the prompts can include text-based queries, structured data inputs, or application programming interface calls that elicit context-specific responses from the AI agent. For example, for a behavioral or performance test, the system can transmit a verification message to the AI agent containing structured input data corresponding to a simulated scenario, such as a patient intake inquiry, a financial compliance check, or a transaction authorization, among others. The AI agent can return outputs to the credentialing system through a response channel established during the initialization phase, allowing the system to record outputs for scoring according to the predefined rubric criteria of the evaluation suite. The credentialing system can generate one or more prompts after retrieving the test suite associated with a credential request and prior to performing evaluation scoring. In some implementations, prompt generation can occur dynamically as the credentialing system executes phase two of the credentialing workflow. For example, the test definitions stored in the test database 724 can include structured templates and contextual parameters that the test executor 720 uses to assemble prompt queries directed to the verified artificial intelligence (AI) agent 512. In some implementations, the prompt templates can specify a question pattern, a context variable set, and a response type indicator that determine how input stimuli are generated for functional evaluation. For example, in a medical domain evaluation, the prompt content can include a mock patient dialogue containing symptom descriptions that require the AI agent 512 to extract key terms or formulate diagnostic observations according to domain-specific criteria.

Responses from the AI agent can be evaluated according to a rubric defining quantitative and/or qualitative metrics associated with individual tests. In some implementations, the rubric can include measurable performance metrics such as accuracy, precision, F1 score thresholds, or bias indices that correspond to specific evaluation categories. For example, expected outputs can be defined in the test database and can be compared with responses collected by the test executor to compute an accuracy ratio or bias variance. The evaluation can occur after responses are received from the AI agent, and can proceed across multiple test sets to complete scoring for each evaluation category. In some implementations, the scoring engine can compute metric values for each test category and update intermediate results associated with the evaluation sequence before the final credentialing determination is made.

At 830, the method 800 can include generating a verifiable credential based on the evaluation indicating that the AI agent 512 or exceeds thresholds defined by the rubric. In some implementations, a structured data object containing the evaluation results can be assembled, and a cryptographic signing process can be applied to the object using a key stored in a secure key management service. For example, a data structure can be generated that includes fields for the credential identifier 708, evaluation suite identifiers, performance summaries, hashes of result logs, and/or timestamps corresponding to the evaluation cycle. The verifiable credential can be recorded in a credential registry accessible to licensed verification, licensing, or insurance entities. In some implementations, the verifiable credential 732 can be encoded with metadata describing the test identifiers, rubric thresholds, and AI agent fingerprint used during the evaluation, providing a transparent and machine-readable record of the credentialing outcome.

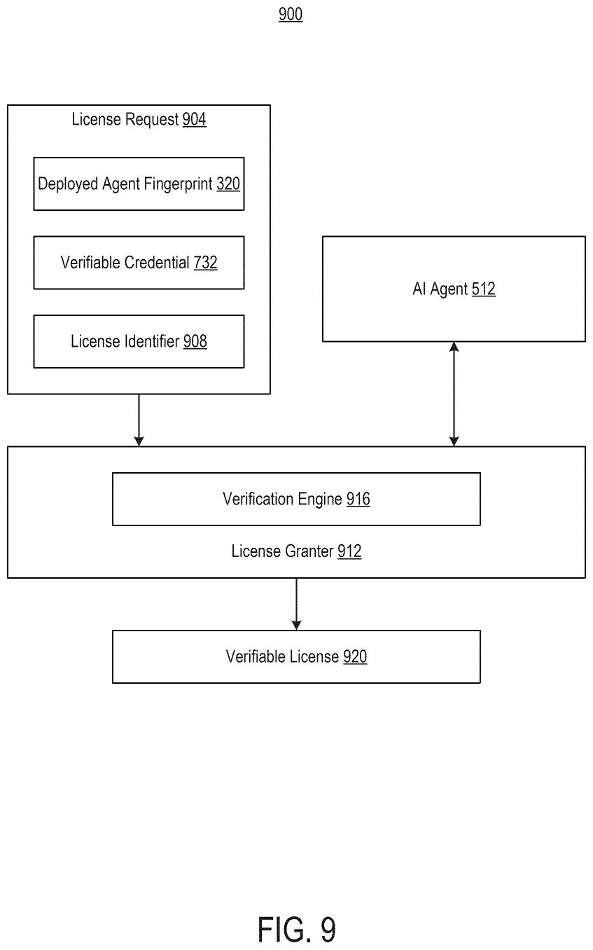

Referring now to FIG. 9, illustrated is a block diagram of an example system 900 to validate deployed AI agents, such as to facilitate granting of cryptographically verifiable licenses to AI agents. The system 900 can include or be coupled with one or more components of various systems described herein, such as to be communicably coupled with one or more of the systems 100, 300, 500, 700 (e.g., to communicate with the instance of the AI agent 512 as deployed by the system 500 and/or receive the verifiable credential 732 from the system 700). In brief overview, the system 900 can include a license granter 912 that can receive a license request 904 that includes the deployed agent fingerprint 320 for an instance of the AI agent 512, the verifiable credential 732 for the AI agent 512, and a license identifier 908 that identifiers a license being requested for the AI agent 512. The license granter 912 can include a verification engine 916 that can verify one or more components of the AI agent 512 (e.g., using data provided in the license request 904 and/or via testing of the AI agent 512) to determine to generate a cryptographically verifiable license 920.

Referring to FIG. 9 in further detail, the system 900 (e.g., the license granter 912) can receive a license request 904. The license request 904 can include a data object or message transmitted from a requesting entity to a licensing entity (e.g., an entity that manages or maintains the system 900). The license request 904 can be a request for the system 900 to generate the cryptographically verifiable license 920 for operation of the AI agent 512 according to one or more criteria, such as criteria of a jurisdiction (e.g., geographic or governmental region) and/or scope of practice (e.g., one or more tasks or uses of the AI agent 512). In some implementations, the license request 904 can be transmitted by a customer or manufacturer computing system to a license granting organization (LGO) endpoint that hosts the licensing service. For example, a customer computing system (e.g., system 500) can generate a transmission request containing metadata such as version tags, scope descriptors, and digital signatures, which the LGO system can receive at a predefined interface to initiate the licensing workflow.

The license request 904 can identify the AI agent 512 under evaluation, such as by including a unique identifier corresponding to or derived from the deployed agent fingerprint 320. The license request 904 may include multiple component fields, such as to include one or more of the deployed agent fingerprint 320, the verifiable credential 732, and the license identifier 908. For instance, the license request 904 may include JSON-formatted fields carrying encoded fingerprint and credential data secured with digital signatures.

The license request 904 can include a license identifier 908. The license identifier 908 can be a token, string, or code referencing a specific agentic license that the AI agent seeks to obtain. For example, the license identifier 908 can take the form of a unique alphanumeric code corresponding to a jurisdictional license such as “Licensed AI Intake Assistant-California”. The license identifier 908 can specify eligibility criteria used by the verification engine 916 and license granter 912 during verification of prerequisite credentials for the license. The license identifier 908 can operate as a reference token that enables retrieval of prerequisite credential identifiers from a licensing database. In some implementations, the license identifier 908 can be appended to the license request 904 during submission to the licensing entity to specify which verifiable credential 732 is referenced for targeted authorization. For example, a requesting system can append the license identifier 908 to the body of the license request 904 as a digitally encoded field that accompanies signature metadata or digital signature verification data transmitted over an application programming interface endpoint during submission. In some implementations, the appended identifier can allow the verification engine 916 to access a stored mapping between license identifiers and required credential identifiers in order to validate the verifiable credential 732 before the license granter 912 generates a verifiable license 920.

Referring further to FIG. 9, the system 900 can include at least one license granter 912. The license granter 912 can include one or more processors, algorithms, rules, heuristics, logic, policies, databases, lookup tables, machine learning models, functions, or combinations to perform operations such as verifying the signature of the verifiable credential 732 based at least on a key (e.g., public key) of the credential granting organization that generated the verifiable credential 732, verifying that the instance of the AI agent 512 is consistent with the deployed agent fingerprint 320, and/or generating verifiable licenses for operation of the AI agent 512.

In some implementations, the license granter 912 includes a verification engine 916, which can verify AI agents 512 against corresponding deployed agent fingerprints 320, and can verify that the verifiable credential 732 for the AI agent 512 is valid. The verification engine 916 can be or include one or more functions of the instance verifier 716 described with reference to FIG. 7.

The verification engine 916 can extract cryptographic data (e.g., hashes of the components represented in the deployed agent fingerprint 320) from the deployed agent fingerprint 320 and can validate the integrity of each component hash identified therein. In some implementations, the verification engine 916 can verify that each hash element referenced in the deployed agent fingerprint 320 accurately corresponds to the canonical manifest of the AI agent 512 as maintained by the licensing entity. For example, the verification engine 916 can access a canonical repository of reference manifests associated with the AI agent 512, recompute respective component hashes from stored configuration data, and compare the resulting hash values to those declared in the submitted deployed agent fingerprint 320. In some implementations, the verification engine 916 can invoke one or more cryptographic or attestation routines to further verify that the deployed runtime environment of the AI agent 512 conforms to the declared configuration. For example, the verification engine 916 can issue an attestation request to a trusted execution environment (TEE) instance associated with the AI agent 512, receive a signed attestation report indicating enclave identity and measurement data, and determine that the reported evidence corresponds to the fingerprint submission before transmitting verification results to the license granter 912.

The verification engine 916 can verify the verifiable credential 732 included in the license request 904 by performing a digital signature validation process to determine that the credential was issued by an authorized credential granting entity. The verification engine 916 can identify a public key associated with the credentialing entity referenced in the verifiable credential 732, can retrieve the key from a trusted registry, and can apply a cryptographic verification routine to validate the digital signature of the verifiable credential 732. In some implementations, the verification engine 916 can validate additional components of the verifiable credential 732, such as credential identifiers, issuance timestamps, or embedded hashes referencing the deployed agent fingerprint 320. For example, upon extraction of the credential payload from the request, the verification engine 916 can compute a checksum over the credential content and compare it with a signature-derived value to confirm that the credential has not been modified after issuance. The verification engine 916 can output a credential verification status used by the license granter 912 to initiate subsequent evaluation of the AI agent 512 for license issuance.

In some implementations, the verification engine 916 can determine to grant the requested license after verifying that an identifier embedded within the verified credential 732 matches one or more prerequisite credential identifiers associated with the license identifier 908. For example, the verification engine 916 can compare the credential identifier to a record stored in a licensing database that defines credential prerequisites tied to specific jurisdictional or functional scopes of practice. In some implementations, when the identifiers match, the verification engine 916 can compile cryptographic elements that bind the verified credential 732, the deployed agent fingerprint 320, and the license identifier 908 into a single digital artifact. For example, the verification engine 916 can compute a hash of the deployed agent fingerprint 320, concatenate the hash with a reference to the verified credential 732, and sign the composition using a private key of the licensing entity. The resulting output can form a digitally signed data structure such as a JSON Web Token or a linked-data credential formatted to include a unique license identifier, scope of practice conditions, jurisdictional parameters, and an expiration timestamp associated with the authorization.

Referring further to FIG. 9, the license granter 912 can generate a verifiable license 920 based at least on the verification engine 916 verifying the deployed agent fingerprint 320 and/or the verifiable credential 732, such as by confirming credential validity and fingerprint consistency. For example, the license granter 912 can generate a verifiable license 920 in response to successful validation of the deployed agent fingerprint 320 and the verifiable credential 732 returned by the verification engine 916.

The verifiable license 920 can be a digitally signed data object that provides cryptographic proof of authorization for an artificial intelligence (AI) agent to operate within a specified jurisdiction or scope of practice. The verifiable license 920 can include data elements such as a license identifier, a hash of the deployed agent fingerprint 320, a reference to the verifiable credential 732, and/or a digital signature of the licensing entity. In some implementations, the verifiable license 920 can serve as a machine-readable token that external systems can independently verify using a public key associated with the licensing entity.

In some implementations, the license granter 912 can store or retain the generated verifiable license 920 for subsequent validation requests and transmit a copy of the same to the requesting entity through a secure interface. For example, the license granter 912 can return the verifiable license 920 as an encoded payload in an application programming interface response directed to a customer computing system or a manufacturer system that initiated the license request 904. The verifiable license 920 can include the public key of the licensing entity so that third-party systems can validate its authenticity independently through public key infrastructure. In some implementations, the license granter 912 can apply an asymmetric digital signature using cryptographic keys managed within a cloud key management service to guarantee verifiability across distributed systems. For example, the license granter 912 can generate the verifiable license 920 and transmit it as an encrypted JSON or tokenized object through a hypertext transfer protocol secure (HTTPS) channel, allowing external entities to parse and validate the embedded hash and jurisdictional metadata included within the signed data object.

The license granter 912 can periodically monitor an executing instance of the AI agent 512 to re-evaluate the validity of the verifiable license 920. The license granter 912 can monitor the AI agent 512 responsive to trigger conditions such as a request to verify the license, a schedule of re-evaluation, or expiration (or a period before expiration) of the verifiable license 920. In some implementations, the license granter 912 can request updated operational data or component hashes from the AI agent 512 at defined intervals to determine whether the deployed configuration remains consistent with the deployed agent fingerprint 320 associated with the verifiable license 920. For example, the license granter 912 can transmit a verification request to a runtime endpoint of the AI agent 512, receive a collection of integrity parameters describing its currently loaded data, model, or software digests, and compare those values with the original fingerprints validated during license issuance. In some implementations, the license granter 912 can evaluate any detected variation in the configuration parameters against a set of licensing conditions encoded within the verifiable license 920 to determine whether continued authorization is warranted. For example, if a monitored hash value or dependency version deviates from the previously verified composition beyond a defined tolerance threshold, the license granter 912 can initiate a re-evaluation process to determine whether the verifiable license 920 remains valid or should be revoked. The periodic monitoring cycle can therefore maintain functional alignment between the licensed scope recorded in the verifiable license 920 and the continuously operating state of the AI agent 512 (and may provide a technical process to allow for variation within operation of the AI agent 512 that is still consistent with the requirements for the verifiable license 920).

The license granter 912 can impose a time limit or geographic limit on the verifiable license 920 by embedding constraint parameters into the license data structure and linking those parameters to validation triggers executed by the verification engine 916. In some implementations, the license granter 912 can insert a timestamp field and a jurisdictional code field that define the temporal and spatial boundaries within which the AI agent 512 may operate. For example, the timestamp field can indicate an expiration date (e.g., time stamp of expiration), and the jurisdictional code field can reference a geographic region or regulatory identifier such as a state, province, or country code. The verification engine 916 can periodically or continuously evaluate status data provided by the AI agent 512, including a time parameter and a deployment location indicator, against the limits defined in the verifiable license 920. In some implementations, when the temporal value exceeds the set expiration timestamp or the geographic indicator falls outside the encoded jurisdictional bounds, the license granter 912 can generate a revocation command that invalidates the verifiable license 920 and halts related operational authorizations for the AI agent 512. For example, the revocation command can invoke a license status update within a license registry used by dependent systems to deny further operation of the AI agent 512 beyond its licensed time window or authorized jurisdiction. The license granter 912 can thereby enforce real-time adherence of the AI agent 512 to both temporal and territorial licensing conditions defined in the verifiable license 920.

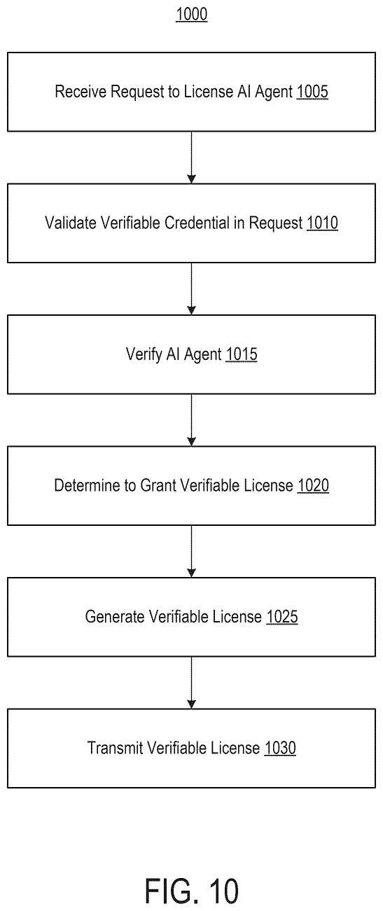

Referring now to FIG. 10, illustrated is a flow chart of an example method 1000 of issuing a verifiable license to an artificial intelligence (AI) agent. The method 1000 can be executed, performed, or otherwise carried out by any computing system or entity described herein, such as a licensing system or license granting organization. In brief overview of the method 1000, the method 1000 can include receiving a request to license an AI agent (1005), validating a verifiable credential in the request (1010), verifying the AI agent (1015), determining to grant a verifiable license (1020), generating the verifiable license (1025), and transmitting the verifiable license (1030).

At 1005, the method 1000 can include receiving a request to license an artificial intelligence (AI) agent. The licensing entity can receive the request from a customer computing system or a manufacturer computing system submitting a deployed agent fingerprint of the AI agent, a verifiable credential for the AI agent, and a license identifier indicating a license being requested for generation for the AI agent. In some implementations, the licensing system can receive the request as a digitally signed data message that carries encoded objects transmitted through an application programming interface endpoint associated with the licensing entity. For example, a customer computing system can call a defined endpoint that transmits a structured payload including the deployed agent fingerprint, the verifiable credential, and the license identifier to initiate the licensing workflow. In some implementations, receipt of the request can trigger initialization of a verification process that cross-references stored credential metadata associated with the identified agent. For example, upon detecting a valid license identifier, the licensing system can initiate a lookup operation to retrieve credential schema information corresponding to the requested license category from an internal credential registry.

At 1010, the method 1000 can include validating the verifiable credential in the request. The validation can be performed by a verification engine of the licensing entity and can include cryptographically verifying the digital signature embedded in the verifiable credential using a public key associated with a credential granting organization that issued the credential. In some implementations, the verification engine can obtain the public key from a distributed key registry or a cloud-based key management service and can apply an asymmetric signature verification algorithm to the received credential payload. In some implementations, the validation can confirm authenticity of the issuer, verify a timestamp of issuance, and confirm that the credential has not expired or been revoked. For example, the verification engine can identify a credential identifier stored in the credential payload and compare it with a registry entry associated with the issuing credential granting organization before proceeding to agent verification.

At 1015, the method 1000 can include verifying the AI agent, such as to verify that the AI agent as deployed matches the deployed agent fingerprint. The verification can be executed by the verification engine of the licensing entity and can cryptographically confirm that the deployed instance of the AI agent corresponds to the declaration represented by the deployed agent fingerprint. In some implementations, the one or more component hashes associated with the agent, such as hashes for model files, data resources, or configuration manifests, can be recomputed and can be compared against corresponding hash values in the deployed agent fingerprint. For example, the verification engine can access manifests stored in a repository associated with the AI agent and generate a hash digest over the manifest contents to identify any divergence from the validated configuration. In some implementations, the verification engine can perform remote attestation or trusted execution environment interrogation to confirm the runtime environment identity. For example, the verification engine can issue a cryptographic challenge to a secure enclave hosting the AI agent and can evaluate a signed attestation response to validate that the runtime measurement values match those encoded in the deployed agent fingerprint.

At 1020, the method 1000 can include determining to grant a verifiable license. The determination can be executed based on verification results received from the verification engine. In some implementations, an identifier embedded in the verified credential can be evaluated against one or more prerequisite credential identifiers associated with the license identifier. For example, a policy database that maps license identifiers to permissible credential identifiers can be accessed, and can be used to verify that the credential identifier of the AI agent meets eligibility criteria for the requested jurisdiction or scope of practice. In some implementations, a ruleset that evaluates correlations among credential identifiers, jurisdictional tags, and scope metadata can be used to determine whether the credential hierarchy requirements are satisfied. For example, a ruleset may specify that a credential labeled “Certified AI Clinical Intake Assistant—Level 2” must be verified before issuance of a license labeled “Licensed AI Intake Assistant—California.”

At 1025, the method 1000 can include generating a verifiable license. The license can be generated to encode authorization data into a digitally signed object. In some implementations, the license can be generated as a digital object that includes a license identifier, a hash of the deployed agent fingerprint, and/or a reference to the validated verifiable credential. The license can be generated to include authorization metadata such as jurisdictional constraints, expiration timestamps, and defined scope-of-practice tags. In some implementations, the verifiable license can be signed using a private key managed within a cloud key management service before transmitting the license back to the requesting entity. For example, the digital signature can encapsulate all concatenated data fields so that downstream systems can validate the authorization parameters using the corresponding public key of the licensing entity.

At 1030, the method 1000 can include transmitting the verifiable license. The transmission can be performed by the licensing system that generated the verifiable license and can deliver the signed license to the requesting entity identified in the received request. In some implementations, the verifiable license can be transmitted through a secure application interface endpoint using a hypertext transfer protocol secure channel or a blockchain-based publication mechanism. For example, the licensing entity can transmit a response message that includes the verifiable license with associated cryptographic signature metadata and a public key identifier that allows independent verification. In some implementations, the transmission process can generate an acknowledgment message that references a transaction identifier associated with the license request. For example, upon successful transmission, the requesting system can receive the verifiable license as part of an application programming interface response that provides the tokenized license object, the embedded signature field, and jurisdictional constraint metadata for subsequent verification.

The method can include periodically re-evaluating the artificial intelligence (AI) agent to modify or update an existing verifiable license. In some implementations, the licensing entity can initiate re-evaluation cycles at defined temporal intervals or upon detection of modifications to an underlying deployed agent fingerprint. For example, the licensing system can retrieve operational parameters associated with the currently licensed instance of the AI agent and compare them with parameters declared in the previously validated configuration to determine whether any re-licensing criteria are triggered. In some implementations, the verification engine can execute one or more verification routines similar to those performed during initial licensing to verify continued conformity with prerequisite credentials and jurisdictional requirements. For example, the verification engine can assess updated model hashes, dependency manifests, or environment identifiers to identify variances affecting the scope of authorization. Based on the re-evaluation outcome, the licensing entity can update the cryptographically verifiable license by generating a revised signature that encodes new expiration parameters, scope conditions, or credential references while maintaining linkage to the verified deployed agent fingerprint.

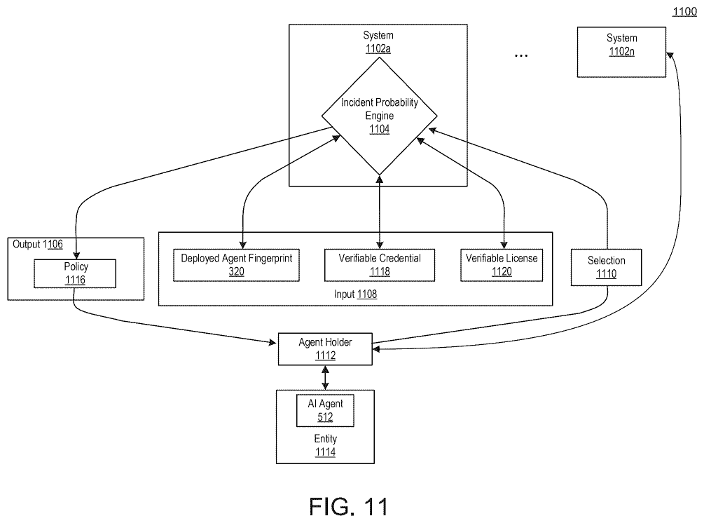

Referring now to FIG. 11, in brief overview, illustrated is a block diagram of an environment 1100 for AI agent credentialing, such as to credential an AI agent according to a policy regarding authorized deployment of the AI agent. The environment 1100 can include one or more systems 1102a-1102n (collectively referred to herein as systems 1102), an entity 1114, and an agent holder 1112. Each of the systems 1102 can include an incident probability engine 1104. The entity 1114 may include the AI agent 512 and/or include or correspond to a system maintained by an entity, such as a manufacturer or customer, that can deploy the AI agent 512. The system 1102a may generate output 1106 based on input 1108. The input 1108 may include the deployed agent fingerprint 320, a verifiable credential 732, and a verifiable license 920. The output 1106 may include a policy 1116. Based on the output 1106, the agent holder 1112 can generate a selection 1110.

Referring to FIG. 11 in further detail, each of the systems 1102 can represent a computing system that can evaluate the AI agent 512. For example, the systems 1102 may correspond to a platform that can facilitate AI agent 512 evaluation (e.g., separately from systems that are deployed by insurers) and/or can correspond to insurers that can provide risk mitigation and/or insurance coverage for the AI agent 512. As an example, based on the input 1108, the system 1102a can generate the policy 1116 (e.g., policy data structure 1116). The policy 1116 can represent and/or be a data structure that represents (e.g., in one or more fields of the data structure) an insurance plan offered by the system 1102a for the AI agent 512. The systems 1102 can transmit and receive data from the entity 1114. For example, the systems 1102 can include components such as processors, memory, and/or application programming interfaces (APIs). The systems 1102 may securely transmit and receive data from the entity 1114 via the components. For example, the systems 1102 and the entity 1114 can exchange cryptographically verifiable data packets through the computing components. In this example, the cryptographically verifiable data packets may identify the AI agent 512. Cryptographically verifiable data packets may refer to data packets that include digital signatures (e.g., cryptographic signatures) that can be used for verification. As an example, the systems 1102 may receive input 1108. The input 1108 may be transmitted to the systems 1102 from the entity 1114. Based on data included in the input 1108, the systems 1102 can generate the policy 1116. In some examples, the systems 1102 can communicate with services. For example, the systems 1102 can communicate with services that perform tasks such as cryptographic validation, aggregation, and/or the like. The systems 1102 may maintain a record of invoked services. For example, the systems 1102 may log service invocations within an auditable ledger. An auditable ledger may be any ledger that includes an immutable record (e.g., blockchain ledger, cryptographically secured ledger, and/or the like).

The incident probability engine 1104 can include one or more models that can estimate a probability of one or more incidents associated with the AI agent 512. For example, the incident probability engine 1104 can apply statistical and/or machine learning methods to evaluate the probability of incident associated with AI agents. Incidents may refer to a failure of the AI agent 512, e.g., with respect to one or more performance criteria, such as failing to meet the performance criteria. As an example, probability of incident may refer to probability of the AI agent 512 generating an output that fails to meet one or more accuracy criteria. Based on one or more components of the input 1108, the incident probability engine 1104 can generate a score corresponding to the AI agent 512. The score may represent the probability and/or severity of incident. As an example, the incident probability engine 1104 can assign the AI agent 512 to a class within a set of classes based on the input. Each class in the set of classes may correspond to different levels (e.g., ranges, such as subranges of scores among a total range of possible scores) of probability of incident. The incident probability engine 1104 may execute one or more models to generate the score. The models may evaluate data from the input 1108 that includes the AI agent 512's performance metrics on representative datasets to generate the score. For example, the models can analyze historical accuracy rates, error occurrences, and anomaly detection results derived from benchmark test sets to quantitatively assess the likelihood and/or potential severity of incidents associated with deployment of the AI agent 512. In some examples, the incident probability engine 1104 can estimate the probability of incident based on detailed performance metrics. For example, the incident probability engine 1104 can evaluate performance metrics (e.g., evaluation data) including historical test categories, weighted scores from evaluation rubrics, and/or hashed logs of functional test outcomes to generate an estimation of the probability of incident for the AI agent 512. In some examples, the incident probability engine 1104 may generate the probability of incident by weighting data in the input 1108 based on a defined evaluation rubric. As an example, the incident probability engine 1104 may assign greater weight to safety and bias assessment results relative to other performance metrics when calculating the probability of incident based on the evaluation rubric. The weighting based on the evaluation rubric may allow the incident probability engine 1104 to generate the probability of incidence based on a defined importance of the various performance metrics. In some examples, the system 1102a can generate the policy 1116 based on an incident probability score generated by the incident probability engine 1102a. For example, based on determining that the AI agent 512 is associated with a relatively high probability of incident based on the output of the incident probability engine 1102a, may generate the policy 1116 with reduced coverage parameters and/or increased cost.

The input 1108 can include data associated with validation of the AI agent 512. The input 1108 can be transmitted in structured payloads (e.g., JSON objects, XML objects and/or the like). The input 1108 can include digital signatures that can be used to confirm authenticity of associated data. The input 1108 may be validated by the systems 1102. For example, the systems 1102 can validate digital signatures of one or more components of the input 1108. The validated input 1108 can then be processed within the incident probability engine 1104 to generate output representing associated with a probability of incident corresponding to the AI agent 512. The input 1108 may associate the information with the AI agent 512 through the inclusion of the deployed agent fingerprint 320. For example, the deployed agent fingerprint 320 may include a cryptographic hash identifier that corresponds to the AI agent 512.

The input 1108 can include the verifiable credential 732. The verifiable credential 732 can represent a digitally signed attestation of the performance of the AI agent 512. The verifiable credential 732 may be generated by a credential organization. The credential organization may be any entity that can issue credentials. As an example, a credential organization for healthcare AI agents may be a recognized medical certification body, such as the American Medical Association (AMA). In an example, the verifiable credential 732 can include the results of functional evaluation. Results of functional evaluation can include bias, safety, and/or efficacy metrics. In an example, the results of the functional evaluation may be encoded within a machine-readable metadata schema (e.g., JSON object, and/or the like). The verifiable credential 732 may serve as input to the incident probability engine 1104. For example, the verifiable credential 732 may be parsed to extract evaluation data. The incident probability engine 1104 may generate a score for the AI agent 512 based on the evaluation data. The score can be used to select the policy 1116 for the AI agent 512. In some examples, the systems 1102 may verify authenticity of the verifiable credential 732 based on a digital signature. For example, the systems 1102 can verify authenticity of the verifiable credential 732 based on verifying the digital signature using a public key. The systems 1102 can retrieve the public key from a secure registry. The public key may be retrieved from the credential organization that generated the verifiable credential 732. The systems 1102 can then validate the integrity of the verifiable credential 732 by confirming that the cryptographic signature matches the credential content. As an example, the digital signature may use a JSON Web Token (JWT) format signed using asymmetric cryptography (e.g., Elliptic Curve Digital Signature Algorithm (ECDSA), and/or the like).

The input 1108 can include a verifiable license 920. The verifiable license 920 can represent an authorization artifact issued by a license organization. The license organization may grant legal permission for operation of the AI agent 512. As an example, a license organization for healthcare AI agents may be a medical board responsible for licensing healthcare practitioners (e.g., the California Medical Board). The verifiable license 920 may certify that the AI agent 512 holds legal permission to perform a task within a jurisdiction. As an example, the verifiable license can certify that the AI agent 512 can perform patient intake operations within a specified jurisdiction. In some examples, the verifiable license 920 may include a digital signature based on which the verifiable license 920 can be verified. The digital signature may use a public key of the license organization. Based on the public key and data included within the verifiable license 920, the systems 1102 can verify that the verifiable license 920 is authentic. As an example, the systems 1102 can verify authenticity by recalculating a local hash of the payload of the verifiable license 920 and/or comparing it to a referenced digest contained within the license's structured data. The referenced digest may be a hash value included with the verifiable license 920.

The system 1102a can generate the output 1106. The output 1106 may include data generated based on the input 1108. For example, the output 1106 can include the policy 1116 generated for the AI agent 512. The policy 1116 can be or correspond to an offer for insurance coverage. For example, the policy 1116 may include a policy identifier, coverage terms, and/or an identifier of the AI agent 512. Additionally, or alternatively, the policy can include an identifier of the verifiable credential 732, the verifiable license 920, an identifier of the system 1102a, a cryptographic signature, criteria for use, incident probability class (e.g., generated by the incident probability engine 1104), and/or timestamps. The identifier of the AI agent 512 may correspond to the deployed agent fingerprint 1122. Coverage terms of the policy 1116 can specify the entity 1114, applicable time periods, and/or conditions for claims. The coverage terms may be based at least in part on an operating jurisdiction of the AI agent 512 and/or the verifiable license 920. As an example, the coverage terms of the policy 1116 can define coverage duration, intended use cases, expected activity load, or geographic limits, as specified in the received coverage parameters. The policy 1116 can be generated automatically as a machine-readable data object (e.g., JWT) digitally signed with the a cryptographic key of the system. For example, after signing, the system 1102a can transmit the policy 1116 back to the agent holder 1112. The policy 1116 may be transmitted to the AI agent 512 through an authenticated API response.

The agent holder 1112 can represent a computing system that can transmit and receive data associated with the AI agent 512. For example, the agent holder 1112 may be an entity (e.g., such as a customer, manufacturer of record, and/or the like) associated with deployment of the AI agent 512. As an example, the agent holder 1112 may be a healthcare provider or law firm deploying the AI agent 512. The agent holder 1112 may deploy the AI agent 512 in a regulated market. As a result, the agent holder 1112 may request the policy 1116 indicating coverage details for deployment within the regulated market. A regulated market may refer to an industry that uses licensing for approval to operate (e.g., healthcare, financial services, legal practice, and/or the like). The agent holder 1112 may transmit and receive data via API requests. As an example, the agent holder 1112 can transmit API requests to the systems 1102 including the deployed agent fingerprint 1122, verifiable credential 732, and verifiable license 920 to initiate coverage requests. Based on the output 1106, the agent holder 1112 can generate the selection 1110.

The agent holder 1112 can generate the selection 1110 based on selecting a policy generated by one of the systems 1102. The selection 1110 may be generated based on one or more defined rules. The rules may determine a selected policy based on attributes of policies (e.g., cost, coverage, and/or the like). The selection 1110 may be generated based on user input. In an example, the selection 1110 can trigger generation of a digitally verifiable policy credential according to the selected request. For example, in response to receiving a selection of the policy 1116, the system 1102a may assemble and digitally sign the policy 1116 to generate a digitally verifiable policy credential. The digitally verifiable policy credential can be verified based on the digital signature. In some examples, the selection 1110 may invoke an internal function call that triggers storage operations within a compliance registry. For example, the policy 1116 may be stored within the compliance registry in response to the system 1102a receiving the selection 1110.

The entity 1114 may be a computing system that can provide the AI agent 512. For example, the entity 1114 may host the AI agent 512 and/or manage deployed resources of the AI agent 512. The entity 1114 may transmit data packages through secure channels. The entity 1114 may also maintain authorization records linked to the verifiable credential 732 and/or the verifiable license 920. To maintain authorization records, the entity 1114 may synchronize credential and policy updates with compliance repositories (e.g., provided by the license organization, credential organization, and/or the like). In some examples, the entity 1114 may participate in auditing workflows that ensure regulatory adherence. For example, the entity 1114 may generate cryptographically signed audit logs of credential and license usage. The entity 1114 can provide the logs to independent compliance registries associated with deployment of the AI agent 512. In some examples, the entity 1114 may be a computing system of an organization (e.g., hospital, financial firm, and/or the like) or customer that can deploy the AI agent 512 to execute a task. As an example, if the AI agent 512 is configured to execute patient intake tasks, the entity 1114 may be associated with a hospital that can execute the AI agent 512 to execute patient intake tasks for patients of the hospital.

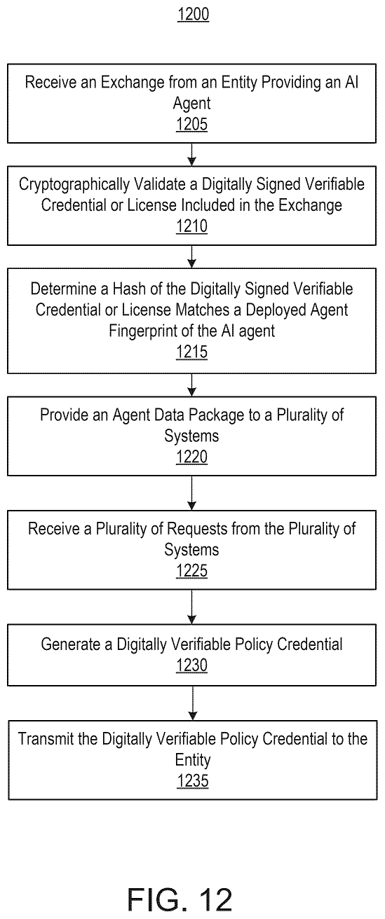

Referring now to FIG. 12, illustrated is a method 1200 of AI agent credentialing. The method 1200 can be executed, performed, or otherwise carried out by any of the computing systems or devices described herein, including, for example, one or more components of the system 1100. In a brief overview of the method 1200, the method 1200 can include receiving an AI agent exchange (1205), validating a verifiable credential or license (1210), determining a hash match to a deployed agent fingerprint (1215), providing an agent data package to a plurality of systems (1220), receiving a plurality of requests from the plurality of systems (1225), generating a digitally verifiable policy credential (1230), and transmitting the digitally verifiable policy credential to an entity (1235).

At 1205, the method 1200 can include receiving a request for credentials and/or licenses of an AI agent (e.g., the AI agent 512 of FIG. 11). The request may be received from an entity (e.g., the entity 1114 of FIG. 11) via an exchange. An exchange may refer to a digital platform that can facilitate secure data transmission. The request may request a digitally verifiable policy credential (e.g., the policy 1116 of FIG. 11). The request may include a digitally signed verifiable credential (e.g., the verifiable credential 732 of FIG. 11) and/or a digitally signed verifiable license (e.g., the verifiable license 920 of FIG. 11) associated with the AI agent. Digitally signed may refer to any data that includes a cryptographic signature that can be used to validate data contents. The request may include a deployed agent fingerprint (e.g., the deployed agent fingerprint 320 of FIG. 11) corresponding to the AI agent. The request may include one or more parameters for coverage. For example, the request can include parameters such as duration of coverage, intended use case, expected activity load, and/or geographic region of operation. In some examples, the one or more processors may receive the request in response to an API call including input (e.g., the input 1108 of FIG. 11). As an example, an agent holder (e.g., a customer or manufacturer of record) can initiate an API call that transmits a deployed agent fingerprint, verifiable credential, and verifiable license of the AI agent to the exchange. The one or more processors may receive the request based on the API call. For example, the API call may trigger a workflow associated with generation of the digitally verifiable policy credential.

In some implementations, the parameters for coverage can include duration, intended use case, expected activity load, or geographic region of operation. Duration may define hours, days, or months of active coverage. Intended use case may define task categories such as clinical intake, document review, or financial processing. Expected activity load may describe anticipated task volume or interaction frequency. Geographic region of operation may identify jurisdictions where the AI agent is permitted to function.

At 1210, the method 1200 can include validating at least one of the digitally signed verifiable credential or license. The digitally signed verifiable credential and/or the digitally signed verifiable license can be validated using a public key. For example, retrieve the public key may be retrieved from a database associated with a license organization or credential organization that generated the digitally signed verifiable credential and/or the digitally signed verifiable license. The contents of the digitally signed verifiable credential and/or the digitally signed verifiable license may be validated based on the public key. For example, a hash of a data payload of the digitally signed verifiable credential and/or the digitally signed verifiable license may be generated and compared to the generated hash to a corresponding public key. The corresponding credential or license can be validated based on determining that the generated hash matches the corresponding public key.

At 1215, the method 1200 can include determining a hash match of at least one of the digitally signed verifiable credential or the digitally signed verifiable license to the deployed agent fingerprint included in the request. For example, t a hash of the deployed agent fingerprint can be generated. The generated hash can then be compared to at least one of the digitally signed verifiable credential or the digitally signed verifiable license to determine if there is a match between the generated hash and the at least one of the digitally signed verifiable credential or the digitally signed verifiable license. A match may indicate that the digitally signed verifiable credential and/or the digitally signed verifiable license correspond to the AI agent represented by the deployed agent fingerprint. Based on determining that there is a match, the one or more processors may proceed with a workflow associated with the request to generate the digitally verifiable policy credential. The matching operation can be performed using cryptographic functions (e.g., secure hash algorithm (SHA) 256, and/or the like) stored in secured compute instance associated with the one or more processors. In some examples, a log may be maintained hash functions. As an example, both hash inputs and outputs associated with the deployed agent fingerprint, the digitally signed verifiable credential, and/or the digitally signed verifiable license can be logged to a compliance registry for auditing and traceability.

At 1220, the method 1200 can include providing an agent data package to a plurality of systems. For example, the agent data package may be provided to a plurality of systems corresponding to policy credentialing entities. A policy credentialing entity may be any organization that issues and manages policies for incident coverage (e.g., insurance). The agent data package can be provided over a programmatic API to request an offer to provide the digitally verifiable policy credential from each of the plurality of systems. The agent data package can include the validated deployed agent fingerprint, the digitally verifiable credential, the digitally verifiable license, and/or the parameters for the coverage. For example, the agent data package may include the deployed agent fingerprint and at least one of the digitally verifiable credential or the digitally verifiable license. The agent data package can be transmitted after successful hash confirmation of one or more components (e.g., the digitally verifiable credential, and/or the like). Each system may receive a data payload including the agent data package. The plurality of systems can each provide the agent data package to an internal model (e.g., the incident probability engine 1104 of FIG. 11). The internal model can determine a probability of incident associated with the AI agent.

In some implementations, the agent data package can include evaluation data associated with the AI agent. For example, the agent data package can include quantitative performance metrics such as accuracy, precision, recall, and/or F1 scores. As an example, the evaluation data can include safety assessments, bias assessments, determinations of bias with regulatory standards, logs from testing suites, performance under adversarial conditions, and/or the like. The quantitative metrics may be derived from functional testing. For example, a credential granter (e.g., the credential granter 732 of FIG. 7) and/or a license granter (e.g., the license granter 920 of FIG. 9) can execute functional testing as part of generating the digitally verifiable license and/or the digitally verifiable credential. In some examples, the plurality of systems can generate the plurality of requests based at least in part on the evaluation data. For example, the systems can assess probability of incident based on the evaluation data.

In some implementations, the one or more processors may cause at least some of the plurality of systems to store the agent data package in a database. For example, the one or more processors may send a storage command to the plurality of systems through an authenticated network interface. The database may be configured to handle structured metadata associated with the agent data package. Records in the database may include identifiers such as fingerprints, credential hashes, and/or timestamps. In an example, the storage command may specify database schema parameters defining required fields. In some examples, the database may be immutable. For example, the database may maintain immutability by versioning each insert operation. In some examples, the one or more processors may define access policies. The access policies may restrict write permissions to authorized systems (e.g., the plurality of systems). The database may support queries for verification purposes. As an example, a credential organization may query the database to verify compliance history of a particular AI agent. The stored information in the database may therefore later assist in monitoring the lifecycle of an AI agent deployment across systems.

In some implementations, the entity may store the agent data package in a compliance registry. For example, the one or more processors may transmit instructions to the entity to record at least part of the agent data package. The compliance registry may maintain a secure and auditable record for regulatory use. The stored information may be retained for verification or future audits. The registry may be structured using database tables and/or distributed ledger technology. The one or more processors may define retention policies. For example, automatic updates may be triggered when a new digitally verifiable credential is used. The stored data in the compliance registry may be accessed by authorized parties through authenticated interfaces (e.g., secure APIs, and/or the like). In some examples, one or more operations may be triggered in response to a change in state of the registry. A change in state of the registry can include updates to one or more parts of the agent data package stored in the registry and/or timeouts of one or more parts of the agent data package (e.g., expiration of the digitally verifiable credential, and/or the like). For example, updates to the registry may trigger updates to policy. As an example, in response to identifying an update to the registry, the exchange may transmit a request for an updated request to one or more of the systems.

In some implementations, the plurality of systems may evaluate an incident probability associated with the AI agent. For example, the plurality of systems may use data included as part of the digitally verifiable credential and/or the digitally verifiable license to estimate a likelihood of incident. The digitally verifiable credential and/or the digitally verifiable license may include test data comprising results from functional evaluations. Functional evaluations can include performance metrics, safety assessments, bias detection outcomes, and/or compliance test results. The test data can include quantitative scores and/or qualitative reports on the accuracy, robustness under adversarial conditions, adherence to regulatory standards, and/or operational reliability of the AI agent within intended use cases. Based on the test data, the plurality of systems can determine the incident probability. Incident may refer to operational failure. If the AI agent performs patient intake duties, an incident may be the generation of incorrect patient information, failure to accurately capture symptoms, and/or improper handling of sensitive health data. Each of the plurality of systems may apply a probabilistic model to compute severity and/or frequency values of an incident. The systems may assign the AI agent to a class within a defined set of classes based on an output of the model. Each class may correspond to a level of incident probability. In an examples, at least some of the plurality of systems may generate the policy based on the assigned class. For example, the systems may modify policy attributes (e.g., cost, length, scope of coverage, and/or the like) based on the assigned class. As an example, based on determining that the AI agent is assigned to a relatively high-risk class, a system may reduce the scope of the policy.

At 1225, the method 1200 can include receiving a plurality of requests from the plurality of systems. For example, the plurality of requests to provide the digitally verifiable policy credential (e.g., the policy 1116 of FIG. 11) may be received in response to providing the agent data package. The requests to provide the digitally verifiable policy credential may be collected until a stopping condition is met. The stopping condition may be receiving the request to provide the digitally verifiable policy credential from each of the plurality of systems. Each request to provide the digitally verifiable policy credential can include policy attributes. The policy attributes can include a price, coverage terms, and/or digital signatures by the respective system. In some examples, a request to provide the digitally verifiable policy credential may be stored in response to receiving the request to provide digitally verifiable policy credential from a system of the plurality of systems. In an example, the requests may be stored temporarily. For example, requests to provide the digitally verifiable policy credential may be stored until a selection of one of the requests is made.

In some implementations, the requests to provide the digitally verifiable policy credential may include criteria for use. The criteria for use may define when a policy applies, the permissible operational context, and/or the functional boundaries of coverage. For example, criteria may indicate tasks, operating environments, and/or authorized domains permitted under the coverage terms. In some examples, the criteria may indicate time constraints, usage frequency, or jurisdictional limits. The requests to provide the digitally verifiable policy may include standardized metadata values reflecting conditions for acceptable use. In some examples, the criteria for use may be interpreted before a selection is accepted and/or a resulting policy credential is generated. For example, the one or more processors may determine whether the criteria for use aligns with the parameters for coverage indicated by the entity. In response to determining that the parameters for coverage are not compatible with the criteria for use, a selection may be rejected

In some implementations, the entity may present the plurality of requests. For example, the entity to display a graphical interface that shows a subset of available policy offers. The graphical interface may include fields showing insurer names, coverage durations, and/or cost parameters. The graphical interface may be accessible through a browser or dedicated application. The graphical interface may include one or more selectable options. Selectable options may be presented as a structured card or tab. In an example, a user of the entity may transmit a selection of a request of the plurality of requests by selecting one of the selectable options.

In some implementations, one or more of the plurality of requests may be an API request. For example, the request may be transmitted from the corresponding system through a network interface to an endpoint. The one or more processors may receive the API request via the endpoint. An API response can be generated in response to the API request. The API response can include instructions to present at least some of the plurality of requests at a graphical user interface of a user device (e.g., laptop, mobile device, and/or the like) associated with the entity. The API request may include data fields associated with the digitally verifiable credential, digitally verifiable license, and/or identifiers (e.g., the deployed agent fingerprint, and identifier of the entity, and identifier of an AI model associated with the AI agent, and/or the like) associated with an AI agent. In some examples, the API request may occur over secure transport protocols (e.g., HTTPS, and/or the like). In some examples, each API request may be authenticated. For example, each API request may be authenticated by verifying authorization tokens transmitted with the API request. Verifying authorization tokens may include validating token signatures, checking token expiration times, determining whether the token scope authorizes the requested operation, and/or the like.

In some implementations, the plurality of request may be received as part of a competitive bidding process to provide the digitally verifiable policy credential. For example, each request may represent a dynamically generated offer to provide the digitally verifiable policy credential. In an example, the requests may be competitively solicited. For example, at least part of a first request from a first system may provided to a second system. The details of the first request may be shared with the second system to encourage submission of a more competitive or favorable request (e.g., policy credential offer) by the second system. In some examples, the plurality of requests can represent competing offers generated in real-time (e.g., within a defined bidding window). For example, the requests may be received for a defined period of time after an initial solicitation for requests. A request can then be selected (e.g., based on defined rules, input from a suer device, and/or the like) at the end of the defined period of time.

At 1230, the method 1200 can include receiving a selection of a request of the plurality of requests. For example, the system may receive a user choice identifying the selection of the request (e.g., a selected policy offer). The selection can be transmitted by an agent holder (e.g., the agent holder 1112 of FIG. 11). The decision may be determined based on policy attributes. For example, the agent holder may automatically select the request based on defined rules and the policy attributes. As another example, the agent holder may receive user input comprising the selection of the request. In response to receiving the user input, the agent holder can transmit the selection of the request to the one or more processors. The selection may be captured as a structured data event. In an example, the system associated with the request and/or the one or more processors may record the choice in an auditable ledger. The stored record can include timestamps, requester information, and/or selected policy identifiers.

At 1235, the method 1200 can include generating a digitally verifiable policy credential. For example, the digitally verifiable policy credential can be generated based on the selection of the request to provide the digitally verifiable policy credential. The digitally verifiable policy credential may verify insurance of the AI agent. For example, the digitally verifiable policy credential may include the deployed agent fingerprint, a policy (e.g., the policy 1116 of FIG. 1) corresponding to the one or more parameters, and/or the digitally verifiable policy credential digitally signed based on a key of the exchange. In an example, the digitally verifiable policy credential may include timestamps associated with generation of one or more components. The generation process can invoke cryptographic signing of the digitally signed verifiable credential. Cryptographic signing may use a private key associated with the one or more processors. The digitally signed verifiable credential can then be verified using a public key associated with the one or more processors. The one or more processors may provide (e.g., via an authoritative registry, API endpoint, and/or the like) the public key to external organizations. As an example, a license organization may verify the digitally verifiable policy credential based on retrieving the public key from the one or more processors.

In some implementations, the digitally verifiable policy credential can be a machine-readable data object. For example, the digitally verifiable policy credential can be any type of data object (e.g., JSON Web token, and/or the like). The digitally verifiable policy credential can include a first identifier corresponding to the policy. The digitally verifiable policy credential can include criteria for use of the policy (e.g., geographic limitations, temporal constraints, and/or the like). The digitally verifiable policy can include a second identifier corresponding to a system of the plurality of systems associated with the selected request. The digitally verifiable policy can include an identifier corresponding to the entity providing the AI agent. The digitally verifiable policy can include the deployed agent fingerprint for the AI agent. Identifiers may be universally unique (e.g., the second identifier may be universally unique across the plurality of systems) and/or cryptographic identifiers (e.g., hash values derived from data associated with the digitally verifiable policy credential).

At 1240, the method 1200 can include transmitting the digitally verifiable policy credential to the entity. For example, the digitally verifiable policy credential can be transmitted via an output interface to the entity that submitted the request to obtain the digitally verifiable policy credential. The digitally verifiable policy credential may be transmitted as a digital object for compliance storage or deployment integration. The digitally verifiable policy credential can be transmitted in response to generating the digitally verifiable policy credential. For example, after successful generation of the digitally verifiable policy credential, the one or more processors can push the digitally signed verifiable credential via an HTTPS API response or message delivery queue to a registered endpoint controlled by the entity. In some examples, the one or more processors can store acknowledgment logs or returning confirmation tokens linked to exchange transaction records. In some examples, the transmitted digitally verifiable policy credential can be replicated in archival systems. In these examples, the replicated digitally verifiable policy credential may be used for downstream verification (e.g., by a license organization) and/or renewal of policies. In some examples, the digitally verifiable policy credential can be transmitted to each of the plurality of systems. For example, the digitally verifiable policy credential may be transmitted by the exchange to each of the plurality of systems to maintain synchronized records of digitally verifiable policy credentials.

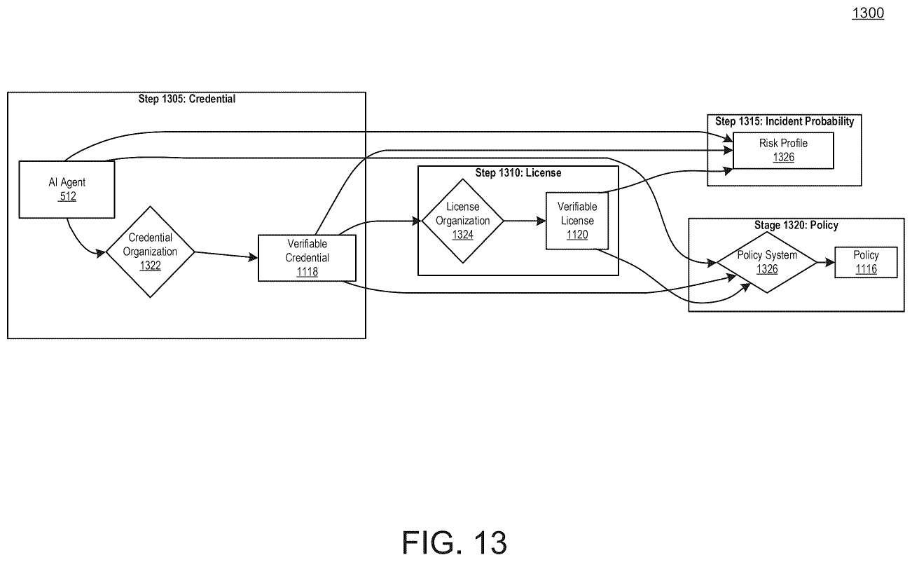

Referring now to FIG. 13, illustrated is a flow diagram of a process 1300 for AI agent credentialing. The process 1300 can be executed, performed, or otherwise carried out by any of the computing systems or devices described herein. In brief overview, at 1305, a credential granter 712 can generate the verifiable credential 732 for the AI agent 512. At 1310, a license granter 912 can generate the verifiable license 920 for the agent 512. At 1315, a risk profile 1326 can be generated for the AI agent 512 based on the verifiable credential 732 and the verifiable license 920. At 1320, the policy 1116 can be generated by the system 1102a based on the AI agent 512, the verifiable credential 732, the verifiable license 920, and the risk profile 1326.

In some implementations, the data flow of the process 1300 may be managed by an exchange system. For example, the exchange system may control routing of a deployed agent fingerprint identifying the AI agent 512, the verifiable credential 732, the verifiable license 920, and the policy 1116 between components associated with the process 1300. In some examples, the exchange system may execute cryptographic validation of received data before transmitting the data to another component. As an example, the exchange system may receive the verifiable credential 732 from the credential granter 712. The exchange system can then execute cryptographic validation of the verifiable credential 732 before transmitting the verifiable credential 732 to the system 1102a. Cryptographic validation may include verifying the digital signature of the verifiable credential 732 and/or the verifiable license 920 using the public key of the issuing entity. As an example, to verify the digital signature of the verifiable credential, the exchange system can decrypt a digital signature included as part of the verifiable credential 732 using the corresponding public key, generate a hash of the verifiable credential 732, and then compare the decrypted value to the generated hash. A match may indicate that the verifiable credential 732 is authenticate. The exchange system can then verify the verifiable credential 732 based on comparing the hash of the verifiable credential 732 to a hash of the deployed agent fingerprint. A match may indicate that the verifiable credential 732 corresponds to the AI agent 512. In some examples, the exchange system may repeat the process of digital signature verifications and comparison of with the deployed agent fingerprint with the verifiable license 920. By combining digital signature verification with hash matching against the deployed agent fingerprint, the exchange system can ensure both the authenticity of the verifiable credential 732 and/or the verifiable license 920 and a correct match between the verifiable credential 732 and/or the verifiable license 920 and the AI agent 512.

At 1305, the credential granter 712 can generate the verifiable credential 732 corresponding to the AI agent 512. The credential granter 712 may verify metadata received from an entity (e.g., the entity 1114 of FIG. 11) associated with the AI agent 512. As an example, a manufacturer of record for the AI agent 512 may generate metadata including component identifiers, evaluation results, deployment configurations, software versions, cryptographic hashes for digital verification, and/or the like corresponding to each core component of the AI agent 512. In some examples, the credential granter 712 may apply cryptographic validation procedures to confirm accuracy of the metadata. For example, the credential organization may perform digital signature verification of at least part of the metadata using a public key associated with the metadata. In some examples, the credential granter 712 may issue a structured record that can be stored in a secure database. The structured record may include a digital signature and/or a timestamp for traceability. In some examples, the credential granter 712 may transmit the verifiable credential 732 through an authenticated interface to authorized entities. For example, the credential granter 712 can transmit the verifiable credential 732 to the system 1102a. In some examples, the credential granter 712 may maintain an internal registry of issued verifiable credentials, including the verifiable credential 732, for auditing and compliance monitoring.

The credential granter 712 may be any organization that can issue verifiable credentials associated with AI agents. For example, the credential granter 712 may be an academic body, a professional association, or a standards consortium. The credential granter 712 may define evaluation criteria for AI agent performance. The credential granter 712 may review evidence provided by a manufacturer of record or customer. The credential granter 712 may operate independently or within a regulatory network. The credential granter 712 may maintain a secure registry for issued credentials and corresponding verification data. The credential granter 712 may provide interfaces for credential lookup, revocation, and/or renewal of the verifiable credential 732. For example, the credential granter 712 can provide an interface that can provide access to public keys for verification of credentials.

At 1310, the license granter 912 can generate the verifiable license 920. The license granter 912 may review evaluation data associated with the AI agent 512. For example, the license granter 912 may receive the verifiable credential 732 that includes evaluation data associated with the AI agent 512. The evaluation data can include output of assessments that evaluate the ability of the AI agent 512 to perform a given task. The license granter 912 may generate the verifiable license 920 based on confirming that the AI agent 512 meets predefined regulatory or operational requirements. For example, based on determining, from the evaluation data, that the AI agent 512 can execute tasks at a threshold proficiency, the license granter 912 can issue the verifiable license 920. In some examples, the verifiable license 920 may include jurisdictional details and/or permitted scope of practice. For example, the license granter 912 can generate a verifiable license 920 that specifies authorized geographic regions, define permitted operational tasks, and/or set temporal validity periods to indicate legally sanctioned boundaries for operation of the AI agent 512. In some examples, the license granter 912 may generate a timestamp and/or expiration record as part of the verifiable license 920. In some examples, the license granter 912 may assign an identifier to each license issued. The identifier may be linked to the deployed agent fingerprint.

In some implementations, the verifiable license 920 can include a digital signature. The digital signature can be used to verify that the verifiable license 920 is authentic. For example, the license granter 912 can generate the digital signature using a private key. Another organization, such as the system 1102a, may use the digital signature to validate integrity and/or authenticity of the license by verifying the signature against the corresponding public key stored in a secure registry. The secure registry may be provided by the license granter 912.

The license granter 912 can be any organization that can issue licenses associated with AI agents. For example, the license granter 912 can be a governmental agency, an industry consortium, a professional board, and/or the like. The license granter 912 may define categories of operational approval for deployed AI agents. As an example, the license granter 912 may be a state medical board that issues licenses authorizing AI agents to perform specific healthcare functions (e.g., clinical patient intake, diagnostic support, and/or the like) within a defined jurisdiction. In some examples, the license granter 912 may manage a registry of licensed AI agents. In these examples, the registry may support lookup, suspension, and/or renewal workflows through digital interfaces.

As an example, the license granter 912 may issue the verifiable license 920 certifying that the AI agent 512 is authorized to perform clinical patient intake activities within a specific jurisdiction after confirming compliance with state medical board regulations. The license granter 912 may confirm that the AI agent 512 is in compliance with state medical board regulations based on determining that the AI agent 512 can perform clinical patient intake activities at a threshold accuracy rate. The license granter 912 may determine that the AI agent 512 can perform clinical patient intake activities based on the verifiable credential 732 issued by a medical professional association (e.g., the National Council Licensure Examination for Registered Nurses). The verifiable credential 732 may include the output of an evaluation that is the same or similar to standardized exams for patient care (e.g., Certified Nursing Assistant (CNA) Competency Exam). The license granter 912 can then generate the verifiable license 920 that indicates the jurisdiction of the given state permitted scope of clinical patient intake activities.

At 1315, the system 1102a can execute one or more models (e.g., the incident probability engine 1104 of FIG. 11) to generate the risk profile 1326. The models can include statistical and/or machine learning models. The models may analyze credential data, license details, and operational metrics included in the verifiable credential 732 and/or the verifiable license 920 to generate the risk profile 1326. As an example, based on evaluation results included as part of the verifiable credential 732, the models can determine a probability of incident when the AI agent 512 is deployed. The models can then generate the risk profile 1326 based on the probability of incident.

The risk profile 1326 can be a representation of an incident probability associated with the AI agent 512. For example, the risk profile 1326 may include quantitative and/or qualitative indicators of a probability of incident. As an example, the risk profile 1326 may provide a numerical score indicating a probability of incident. In some examples, the risk profile 1326 can classify the AI agent 512 into predefined categories (e.g., low, medium, or high) based on statistical analysis of a probability of incident. As another example, the risk profile may include qualitative assessments describing risk factors, operational limitations, and/or contextual considerations. The qualitative assessments may be derived from verified testing and functional evaluations. The indicators may describe likelihood of operational faults and/or deviations during tasks executed by the AI agent 512.

At 1320, the system 1102a can generate the policy 1116. For example, the system 1102a can generate the policy 1116 including an offer of insurance coverage that can be provided based on the risk profile 1326. The system 1102a may assemble policy data elements into a structured data object representing the policy 1116. The policy 1116 may include a coverage identifier, duration, and/or scope of protection. The policy 1116 may be digitally signed using a cryptographic key. The digitally signed version of the policy 1116 may then be transmitted to an agent holder (e.g., the agent holder 1112 of FIG. 11) for review. As an example, the policy 1116 may be transmitted to an organization deploying the AI agent 512 (e.g., a hospital deploying the AI agent 512 for patient intake) as an offer to provide insurance coverage for the AI agent 512. In response to receiving the policy 1116 the agent holder can transmit an acceptance or rejection of the policy 1116. In an example where the agent holder accepts the policy 1116, the system 1102a can finalize proof of coverage under the policy 1116 for the AI agent 512. In some examples, the policy 1116 may be stored in a secure compliance registry. The system 1102a may permit verification of authenticity by authorized entities via the secure compliance registry. As an example, the system 1102a may provide access to the policy 1116 via the secure compliance registry to provide proof of coverage for the AI agent 512 after deployment of the AI agent 512.

Systems and methods as described herein can be implemented by any of various neural networks and/or machine learning models. These can include, for example and without limitation, one or more neural networks (or layers, nodes, weights, and/or biases thereof), convolutional neural networks, recurrent neural networks, attention networks, transformer networks, encoders, decoders, sequence to sequence models, generative models, pretrained models, diffusion models, multimodal models, generative adversarial networks, or various combinations thereof, which may be configured (e.g., trained, fine-tuned, having transfer learning performed, updated or operated by in-context learning, examples, or prompting, etc.) through operations such as supervised learning, self-supervised learning, or unsupervised learning. Systems and methods as described herein can be implemented in any of various artificial intelligence architectures or processing pipelines, including, for example, agentic pipelines, retrieval-based pipelines (e.g., retrieval-augmented generation), or various combinations thereof.

Having now described some illustrative implementations, it is apparent that the foregoing is illustrative and not limiting, having been presented by way of example. In particular, although many of the examples presented herein involve specific combinations of method acts or system elements, those acts and those elements can be combined in other ways to accomplish the same objectives. Acts, elements and features discussed in connection with one implementation are not intended to be excluded from a similar role in other implementations or implementations.

The hardware and data processing components used to implement the various processes, operations, illustrative logics, logical blocks, modules and circuits described in connection with the implementations disclosed herein can be implemented or performed with a general purpose single-or multi-chip processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. A general purpose processor can be a microprocessor, or, any conventional processor, controller, microcontroller, soc (system on chip), som (system on module) or state machine. A processor also can be implemented as a combination of computing devices, such as a combination of a DSP and a microprocessor, a plurality of microprocessors, one or more microprocessors in conjunction with a DSP core, or any other such configuration. In some implementations, particular processes and methods can be performed by circuitry that is specific to a given function. The memory (e.g., memory, memory unit, storage device, etc.) can include one or more devices (e.g., RAM, ROM, Flash memory, hard disk storage, etc.) for storing data and/or computer code for completing or facilitating the various processes, layers and modules described in the present disclosure. The memory can be or include volatile memory or non-volatile memory, and can include database components, object code components, script components, or any other type of information structure for supporting the various activities and information structures described in the present disclosure. According to an exemplary implementation, the memory is communicably connected to the processor via a processing circuit and includes computer code for executing (e.g., by the processing circuit and/or the processor) the one or more processes described herein.

The present disclosure contemplates methods, systems and program products on any machine-readable media for accomplishing various operations. The implementations of the present disclosure can be implemented using existing computer processors, or by a special purpose computer processor for an appropriate system, incorporated for this or another purpose, or by a hardwired system. Implementations within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular can also embrace implementations including a plurality of these elements, and any references in plural to any implementation or element or act herein can also embrace implementations including only a single element. References in the singular or plural form are not intended to limit the presently disclosed systems or methods, their components, acts, or elements to single or plural configurations. References to any act or element being based on any information, act or element can include implementations where the act or element is based at least in part on any information, act, or element.

Any implementation disclosed herein can be combined with any other implementation or implementation, and references to “an implementation,” “some implementations,” “one implementation” or the like are not necessarily mutually exclusive and are intended to indicate that a particular feature, structure, or characteristic described in connection with the implementation can be included in at least one implementation or implementation. Such terms as used herein are not necessarily all referring to the same implementation. Any implementation can be combined with any other implementation, inclusively or exclusively, in any manner consistent with the aspects and implementations disclosed herein.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

Systems and methods described herein can be embodied in other specific forms without departing from the characteristics thereof. Further relative parallel, perpendicular, vertical or other positioning or orientation descriptions include variations within +/−10% or +/−10 degrees of pure vertical, parallel or perpendicular positioning. References to “approximately,” “about” “substantially” or other terms of degree include variations of +/−10% from the given measurement, unit, or range unless explicitly indicated otherwise. Coupled elements can be electrically, mechanically, or physically coupled with one another directly or with intervening elements. Scope of the systems and methods described herein is thus indicated by the appended claims, rather than the foregoing description, and changes that come within the meaning and range of equivalency of the claims are embraced therein.

The term “coupled” and variations thereof includes the joining of two members directly or indirectly to one another. Such joining can be stationary (e.g., permanent or fixed) or moveable (e.g., removable or releasable). Such joining can be achieved with the two members coupled directly with or to each other, with the two members coupled with each other using a separate intervening member and any additional intermediate members coupled with one another, or with the two members coupled with each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling can be mechanical, electrical, or fluidic.

References to “or” can be construed as inclusive so that any terms described using “or” can indicate any of a single, more than one, and all of the described terms. A reference to “at least one of ‘A’ and ‘B’” can include only ‘A’, only ‘B’, as well as both ‘A’ and ‘B’. Such references used in conjunction with “comprising” or other open terminology can include additional items.

Modifications of described elements and acts such as variations in sizes, dimensions, structures, shapes and proportions of the various elements, values of parameters, mounting arrangements, use of materials, colors, orientations can occur without materially departing from the teachings and advantages of the subject matter disclosed herein. For example, elements shown as integrally formed can be constructed of multiple parts or elements, the position of elements can be reversed or otherwise varied, and the nature or number of discrete elements or positions can be altered or varied. Other substitutions, modifications, changes and omissions can also be made in the design, operating conditions and arrangement of the disclosed elements and operations without departing from the scope of the present disclosure.

References herein to the positions of elements (e.g., “top,” “bottom,” “above,” “below”) are merely used to describe the orientation of various elements in the FIGURES. The orientation of various elements can differ according to other exemplary implementations, and that such variations are intended to be encompassed by the present disclosure.

What is claimed is:

1. A system of artificial intelligence (AI) agent credentialing, comprising:
one or more processors to:
receive, by an exchange from an entity providing an AI agent, a request for the AI agent to obtain a digitally verifiable policy credential for coverage of the AI agent, the request comprising a deployed agent fingerprint for the AI agent, at least one of a digitally signed verifiable credential of the AI agent or a digitally signed verifiable license of the AI agent, and one or more parameters for the coverage;
cryptographically validate the at least one of the digitally signed verifiable credential using a public key of a credentialing entity or the digitally signed verifiable license using a public key of a licensing entity;
determine that a hash of the deployed agent fingerprint matches a hash of at least one of the digitally signed verifiable credential or the digitally signed verifiable license;
provide, to a plurality of systems corresponding to policy credentialing entities, an agent data package that comprises the deployed agent fingerprint, the at least one of the digitally signed verifiable credential or the digitally signed verifiable license, and the one or more parameters for the coverage;
receive, from the plurality of systems, a plurality of requests to provide the digitally verifiable policy credential;
receive a selection of a request of the plurality of requests;
generate the digitally verifiable policy credential according to the selected request, the digitally verifiable policy credential comprising the deployed agent fingerprint, a policy corresponding to the one or more parameters, and the digitally verifiable policy credential digitally signed based on a key of the exchange; and
transmit the digitally verifiable policy credential to the entity.

2. The system of claim 1, wherein the one or more processors are to provide instructions to the entity providing the AI agent to store at least a part of the agent data package in a compliance registry.

3. The system of claim 1, wherein the one or more processors are to provide instructions for at least one of the plurality of systems to store the agent data package in a database.

4. The system of claim 1, wherein the one or more processors are to assign the AI agent to a class of a plurality of classes, the plurality of classes corresponding to levels of incident probability, based on the at least one of the digitally signed verifiable credential of the AI agent or the digitally signed verifiable license of the AI agent received from the entity.

5. The system of claim 1, wherein the one or more parameters for the coverage comprise at least one of duration, intended use case, expected activity load, or geographic region of operation.

6. The system of claim 1, wherein the one or more processors are to transmit the digitally verifiable policy credential to each of the plurality of systems.

7. The system of claim 1, wherein each of the plurality of requests to provide the digitally verifiable policy credential comprise criteria for use of the policy.

8. The system of claim 1, wherein the one or more processors are to cause the entity providing the AI agent to present a graphical interface representing at least a subset of the plurality of requests.

9. The system of claim 1, wherein the request is an application programming interface (API) request and the one or more processors are to transmit an API response to the API request comprising instructions to present at least some of the plurality of requests at a graphical user interface (GUI) of at least one user device associated with the entity.

10. The system of claim 1, wherein the digitally verifiable policy credential is a machine-readable data object that comprises at least one of a first identifier corresponding to the policy, criteria for use of the policy, a second identifier corresponding to a system of the plurality of systems associated with the selected request, an identifier of the entity providing the AI agent, or the deployed agent fingerprint for the AI agent.

11. A method for AI agent credentialing, comprising:
receiving, by one or more processors, by an exchange from an entity providing an AI agent, a request for the AI agent to obtain a digitally verifiable policy credential for coverage of the AI agent, the request comprising a deployed agent fingerprint for the AI agent, at least one of a digitally signed verifiable credential of the AI agent or a digitally signed verifiable license of the AI agent, and one or more parameters for the coverage;
cryptographically validating, by the one or more processors, the at least one of the digitally signed verifiable credential using a public key of a credentialing entity or the digitally signed verifiable license using a public key of a licensing entity;
determining, by the one or more processors, that a hash of the deployed agent fingerprint matches a hash of at least one of the digitally signed verifiable credential or the digitally signed verifiable license;
providing, by the one or more processors, to a plurality of systems corresponding to policy credentialing entities, an agent data package that comprises the deployed agent fingerprint, the at least one of the digitally signed verifiable credential or the digitally signed verifiable license, and the one or more parameters for the coverage;
receiving, by the one or more processors, from the plurality of systems, a plurality of requests to provide the digitally verifiable policy credential;
receiving, by the one or more processors, a selection of a request of the plurality of requests;
generating, by the one or more processors, the digitally verifiable policy credential according to the selected request, the digitally verifiable policy credential comprising the deployed agent fingerprint, a policy corresponding to the one or more parameters, and the digitally verifiable policy credential digitally signed based on a key of the exchange; and
transmitting, by the one or more processors, the digitally verifiable policy credential to the entity.

12. The method of claim 11, comprising providing instructions to the entity providing the AI agent to store at least a part of the agent data package in a compliance registry.

13. The method of claim 11, comprising providing instructions for at least one of the plurality of systems to store the agent data package in a database.

14. The method of claim 11, comprising assigning the AI agent to a class of a plurality of classes, the plurality of classes corresponding to levels of incident probability, based on the at least one of the digitally signed verifiable credential of the AI agent or the digitally signed verifiable license of the AI agent received from the entity.

15. The method of claim 11, wherein the one or more parameters for the coverage comprise at least one of duration, intended use case, expected activity load, or geographic region of operation.

16. The method of claim 11, comprising transmitting the digitally verifiable policy credential to each of the plurality of systems.

17. The method of claim 11, comprising providing the digitally verifiable policy credential comprise criteria for use of the policy.

18. The method of claim 11, comprising causing the entity providing the AI agent to present a graphical interface representing at least a subset of the plurality of requests.

19. The method of claim 11, wherein the request is an application programming interface (API) request and the one or more processors are to transmit an API response to the API request comprising instructions to present at least some of the plurality of requests at a graphical user interface (GUI) of at least one user device associated with the entity.

20. A system, comprising:
one or more processors to:
receive a request for a digitally verifiable policy credential for coverage of an AI agent, the request comprising at least one of a digitally signed verifiable credential of the AI agent or a digitally signed verifiable license of the AI agent, and one or more parameters for the coverage;
cryptographically validate the at least one of the digitally signed verifiable credential using a public key of a credentialing entity or the digitally signed verifiable license using a public key of a licensing entity;
provide, to a credentialing system, an agent data package that comprises the at least one of the digitally signed verifiable credential or the digitally signed verifiable license, and the one or more parameters for the coverage;
receive, from the credentialing system, an indication to provide the digitally verifiable policy credential; and
generate the digitally verifiable policy credential according to the indication, the digitally verifiable policy credential comprising a policy corresponding to the one or more parameters.
